SEQUENCE LISTING (19 sequences)

SEQ ID NO: 1 - protein, 17 aa, Neisseria meningitidis
AQAANGGASGQVKVTKA

SEQ ID NO: 2 - protein, 18 aa, Neisseria meningitidis
QPSKAQGQTNNQVKVTKA

SEQ ID NO: 3 - protein, 14 aa, Neisseria meningitidis
PPSKSQPQVKVTKA

SEQ ID NO: 4 - protein, 20 aa, Neisseria meningitidis
AQAANGGASGQVKVTKVTKA

SEQ ID NO: 5 - protein, 12 aa, Neisseria meningitidis
PLQNIQPQVTKR

SEQ ID NO: 6 - protein, 13 aa, Neisseria meningitidis
HVVVNNKVATHVP

SEQ ID NO: 7 - protein, 10 aa, Neisseria meningitidis
YVDEKKMVHA

SEQ ID NO: 8 - protein, 13 aa, Neisseria meningitidis
HYTRQNNADVFVP

SEQ ID NO: 9 - protein, 14 aa, Neisseria meningitidis
YYTKDTNNNLTLVP

SEQ ID NO: 10 - protein, 10 aa, Neisseria meningitidis
YVDEQSKYHA

SEQ ID NO: 11 - protein, 15 aa, Neisseria meningitidis
HFVQQTPKSQPTLVP

SEQ ID NO: 12 - protein, 32 aa, Neisseria meningitidis
SQFKIEDKEKATDEEKNKNRENEKIAKAYRLT

SEQ ID NO: 13 - protein, 42 aa, Neisseria meningitidis
GKFKISDKKPDPNDPTKEIDKDAAEKAKDKKDMDLVHSYKLS

SEQ ID NO: 14 - protein, 42 aa, Neisseria meningitidis
GEFEISGKKKDPKDPKKEIDKTDEEKAKDKKDMDLVHSYKLS

SEQ ID NO: 15 - protein, 39 aa, Neisseria meningitidis
SKFSIPTTEEKNGQKVDKPMEQQMKDRADEDTVHAYKLS

SEQ ID NO: 16 - protein, 42 aa, Neisseria meningitidis
GEFKISDKKPDPTDPKKEIAKTDEEKAKDKIDMDLVHSYKLS

SEQ ID NO: 17 - protein, 40 aa, Neisseria meningitidis
SKFEISDKKKGADGKEVDVDDAQKEKNRANEKIVHAYKLS

SEQ ID NO: 18 - protein, 350 aa, Neisseria meningitidis
IKAGVEGRNIQLQLTEPLQNIQQPQVTKRKSRIRTKISDFGSFIGFKGSEDLGEGLKAVWQLEQDVSVAGGGASQWGNRESFIGLAGEFGTLRAGRVANQFDDASQAIDPWDSNNDVASQLGIFKRHDDMSVSVRYDSPEFSGFSGSVQFVPAQNSKSAYTPAHFVQNKQNKQNQPPTLVPAVVGKPGSDVYYAGLNYKNGGFAGNYAFKYAKHANVGRDAFELFLLGSGSDEAKGTDPLKNHQVHRLTGGYEEGGLNLALAAQLDLSENADKTKNSTTEIAATASYRFGNAVPRISYAHGFDFIERGKKGENTSYDQIIAGVDYDFSKRTSAIVSGAWLKRNTGIGNYT

SEQ ID NO: 19 - protein, 562 aa, Neisseria meningitidis
LDKNWGVRLNSGFASNEGVSYGASVFGKEGNFDGLFSYNRNDEKDYEAGKGFRNVNGGKTVPYSALDKRSYLAKIGTTFGDDDHRIVLSHMKDQHRGIRTVREEFTVGDKSSRINIDRQAPAYRETTQSNTNLAYTGKNLGFVEKLDANAYVLEKERYSADDSGTGYAGNVKGPNHTRITTRGANFNFDSRLAEQTLLKYGINYRHQEIKPQAFLNSKFSIPTTEEKNGQKVDKPMEQQMKDRADEDTVHAYKLSNPTKTDTGVYVEAIHDIGDFTLTGGLRYDRFKVKTHDGKTVSSSNLNPSFGVIWQPHEHWSFSASHNYASRSPRLYDALQTHGKRGIISIADGTKAERARNTEIGFNYNDGTFAANGSYFWQTIKDALANPQNRHDSVAVREAVNAGYIKNHGYELGASYRTGGLTAKVGVSHSKPRFYDTHKDKLLSANPEFGAQVGRTWTASLAYRFQNPNLEIGWRGRYVQKATGSILAAGQKDRKGNLENVVRKGFGVNDVFANWKPLGKDTLNVNLSVNNVFNKFYYPHSQRWTNTLPGVGRDVRLGVNYKF

(19) United States
(12) Patent Application Publication
(10) Pub. No.: US 2017/0326220 A1
(43) Pub. Date: Nov. 16, 2017

(54) MENINGITIS B VACCINE

(71) Applicant: JANSSEN VACCINES & PREVENTION B.V., Leiden (NL)

(72) Inventors: Jan Theunis Poolman, Leiden (NL); Albertus Lambert Zomer, Wijchen (NL)

(73) Assignee: JANSSEN VACCINES & PREVENTION B.V., Leiden (NL)

(21) Appl. No.: 15/527,664

(22) PCT Filed: Nov. 27, 2015
(86) PCT No.: PCT/EP2015/077940

§ 371 Date: May 17, 2017

(30) Foreign Application Priority Data
Nov. 28, 2014 (EP) 14195507.0

(51) Int. Cl. A61K 39/095; C12Q 1/68; A61K 39/00

(52) U.S. Cl. CPC A61K 39/095; C12Q 1/68; A61K 39/00

(57) ABSTRACT

Compositions, including vaccine compositions, are provided comprising outer membrane vesicles (OMVs) from N. meningitidis B (MenB). Also provided are methods of making such compositions comprising growing MenB in culture and isolating the OMVs produced, and the use of MenB vaccine compositions for prevention of meningitis in a subject.

### FIELD OF THE INVENTION

The invention relates to compositions comprising outer membrane vesicles from Neisseria meningitis capsular group B (Men B) bacteria and their use as vaccines. The invention also relates to methods of making such compositions.

### BACKGROUND

Neisseria meningitidis (the meningococcus) is a leading cause of meningitis and septicemia worldwide. Although this Gram-negative diplococcal bacterium is capable of infecting people of all ages, the greatest risk of developing meningococcal disease (MD) is during childhood and in the teenage years. Capsular group B (MenB) strains cause a disproportionate number of cases in infants <1 year, the age group with the highest incidence of disease. Despite advances in healthcare and antibiotic treatments, case fatality rates remain high (10%), even in industrialized countries. Moreover, 30% of survivors are left with devastating, long-term sequelae such as epilepsy, permanent brain damage, deafness and/or limb amputation.

N. meningitidis has been classified into twelve capsular groups based onto the immunochemistry of the capsule. Twelve serogroups are known: A, B, C, X, Y, Z, 29-E, W, H, I, K and L. However, out of the twelve serogroups, six (A, B, C, X, W and Y) account for more than 90% of all disease. Serogroups B, C, W and Y account for majority of cases in developed countries whereas serogroups A and recently emerging serogroup X are the major burden in the sub-saharan part of Africa called also the “meningitis belt”.

Out of all, MenB strains are of particular concern, being responsible for approximately 80% of all meningococcal disease in most developed countries. The rapid onset and severity of bacterial meningitis are major drivers of public fear of this disease.

Currently, polysaccharide or polysaccharide conjugate vaccines with exception of serogroup B and X are in widespread use. Development of a serogroup B polysaccharide-based vaccine has been hindered by the poor immunogenicity of its capsule antigen, and by the potential risk of antibodies cross-reacting with glycosylated human antigens. Thus the development of a comprehensive meningococcal vaccine with the potential to offer protection against group B isolates has focused on sub-capsular antigens such as lipooligosaccharide (LOS) and surface proteins. The design of protein/LOS-based meningococcal vaccines is also complicated, due to the high levels of genetic and antigenic diversity exhibited by these sub-capsular bacterial components.

Despite the challenges, several strain-specific MenB vaccines have been developed in response to prolonged epidemics, such as occurred in Cuba and Norway during the 1980s and, more recently, in New Zealand (1990s) and France (2000s). All these vaccines have been based on wild-type Outer Membrane Vesicles (OMVs) that are naturally produced by meningococci. OMVs are lipid bilayer ‘blebs’ with an average diameter of 50-250 nm, constitutively extruded from the outer membrane of growing Gram-negative bacteria. They generally have a composition similar to the outer membrane of live bacteria, including lipo-oligosaccharide (LOS; the major meningococcal endotoxin) and surface proteins. These vaccines have been generally shown to be immunogenic and protective. However, it has been shown that the induced immune response against OMV is directed primarily against the immunodominant and variable porin A (PorA), which results in a very limited ability to confer protection against heterologous strains. Hence, all OMV-based have been effective in response to local MenB outbreaks, only (see Table 1).

TABLE 1
Meningococcal OMV-based vaccines developed in response to
MenB-caused epidemics (cc—clonal complex)

| | |
|---|---|
| Cuba (1987-1989): caused by | VaMengoc-BC: incidence declined to |
| B:P1.15 (cc32) | 0.08/10⁵ in 2008 |
| Norway (1975-2008) caused by | MenBvac: incidence declined to 1.7/10⁵ |
| B:15:P1.7,16 (cc32) | |
| New Zealand: (1992-2008) | MeNZB: incidence declined to 2.6/10⁵ |
| caused by B:4:P1.7-2.4 (cc41/44) | in 2007 |
| Normandy, France: (2003-2010) | MenBvac: incidence declined to 5.9/10⁵ |
| caused by B:14:P1.7,16 (cc32) | in 2010 |

A further approach based on OMVs, with the aim of increasing strain coverage, was taken by the Netherlands Vaccine Institute (NVI), when developing the vaccines called HexaMen and NonaMen. The data generated for HexaMen during clinical trial [1] demonstrated a good bactericidal activity against multiple MenB strains but only after 4 immunizations, including administration in the second year of life, whilst protection is needed as early as possible in the first year of life. NonaMen is based on the preparation of OMVs from three genetically engineered strains that each express three subtypes of PorA. The strains are further genetically modified by the IpxL1 mutation so that they express penta-acetylated lipid A, which is reported to be much less reactogenic than wild-type LOS [2]. However, as the main component of the vaccine is PorA protein, a limited cross-protection for other minor antigens is obtained by NonaMen. The fact that multiple PorA antigens were expressed on the surface of the same strain is most likely responsible for the uneven immune response obtained by Nonamen [22].

Minor antigens, although much better conserved than immunodominant PorA, have been also investigated for their antigenic potential. Unfortunately, the data obtained with H44/76 strain shown that in the absence of the PorA, minor outer membrane proteins (OMPs) even when overexpressed generate only a weak immune response [23].

A different approach to create MenB vaccines with broad strain coverage based on the use of “universal” antigens was by the application of reverse vaccinology [16]. A problem with this approach is the variability of the identified antigens. The MenB vaccine prepared by Novartis after reverse vaccinology (‘Bexsero’), is composed of three recombinant proteins (including two fusion proteins) and a detergent-extracted outer membrane vesicle (dOMV).

NadA (Neisseria adhesion A) acts as an important adhesin and invasin during colonization and invasion. However, it occurs only in a limited number of pathogenic meningococci, suggesting that its contribution to the vaccine can be limited. Unlike NadA, Neisseria Heparin Binding Protein (NHBP), is present in most isolates of pathogenic meningococci. NHBP is able to bind highly sulphated glycosylates present in mucosal secretion and it was shown to increase MenB serum resistance. The data obtained so far are complex and hence the contribution of the anti-NHBP antibodies raised by the vaccine to the overall protection remains to be seen. Bexsero is composed also of factor H binding protein (fHbp), which binds human fH acting as a negative regulator of alterative pathway of complement. Recruitment of fH decrease C3 decomposition on pathogen surface decreasing complement-mediated lyses. The abundance of fHbp is important for its efficacy. Therefore, the emergence of strains lacking fHbp as well as strains down regulating its expression is of major concern.

This vaccine appears effective, but strain coverage is doubtful and reactogenicity of the vaccine as a result of the presence of LOS is a concern. As yet, Bexsero has been implemented into routine infant immunization schedule only in Quebec (Canada).

The idea of ‘universal’ antigen was persued also by Pfizer, which based its bivalent ‘rLP2086’ MenB vaccine on the factor H binding protein (fHbp); this vaccine appears effective in adolescents and young adults. A study conducted in toddlers showed generally lower SBA responses and reduced cross-reactivity (less than 44.4% with hSBA≧1:4) against fHbp heterologous strains, and it appears that further development of this vaccine in children and infants—an important target population for MenB vaccines—has stopped. In addition, bivalent rLP2086 induces moderate local reactogenicity in adolescents and young adults, with fever reported in up to 10% of subjects.

Previously, researchers in Oxford, [3], [17] analysed a panel of 78 strains, claimed to be representative of the worldwide meningococcal hyper-invasive lineage diversity, and proposed combinations of purified PorA and FetA (FrpB) antigens that would potentially provide broad coverage to MenB in a recombinant vaccine. It was suggested that their ‘standard’ formulation with as few as six sets of PorA epitopes (P1.5-1,2-2; P1.5-2, 16; P1.5, 10; P1.7, 13-1; P1.7-2,4; and P1.20,9) and five FetA epitopes (F1-5, F3-1, F5-1, F3-9, and F5-5), would potentially be broadly protective against meningococcal isolates. Further, they presented an ‘enhanced’ formulation with a seventh set of PorA epitopes (P1.19, 15) with even broader protection against meningococcal isolates. No vaccine composition was actually prepared and tested for functional antibody induction in those studies. In addition, natural folding of the proteins could be compromised when recombinantly expressed outside of their natural environment, which could reduce the actual immunogenicity of the proposed proteins.

Currently there is only one vaccine against serogroup B licensed in Europe (January, 2013), Australia (July, 2013) and Canada (December, 2013). Due to cost effectiveness issues the vaccine has not yet been implemented into routine immunization schedules. Hence, MenB remains a serious health burden, and a good vaccine that gives broad protection is a strong medical need. Vaccines based on non-capsular antigens have been designed, but suffer from the variability of such antigens, which restricts the coverage of these vaccines to a limited number of strains.

Therefore there remains a need for a broadly protective vaccine against MenB.

### SUMMARY OF THE INVENTION

The present invention is based on the finding that, when provided as an outer membrane vesicle composition, a particular sub-set of PorA and FrpB antigens can provide protection against a broad range of different MenB strains while also having low reactogenicity compared to existing MenB vaccines.

In a first aspect the invention provides a composition comprising outer membrane vesicles (OMVs) from at least six different N. meningitidis B (MenB) strains, wherein the at least six MenB strains comprise PorA variable regions 2 (VR2) having the sequences set out in SEQ ID NOs: 6 to 11 and FrpB VRs having the sequences set out in SEQ ID NOs 12 to 17.

Although many hundreds of FrpB (FetA) and PorA variable region variants and sub-variants have been identified, the present inventors have found that a composition comprising outer membrane vesicles from at least six different MenB strains having a particular, optimal, sub-set of variants of the PorA variable region 2 (VR2) and FrpB VRs is capable of providing broad coverage against known pathogenic MenB strains.

Furthermore, compared to recombinant proteins, the present inventors have found that when the FrpB (FetA) and PorA antigens are administered in the natural membrane environment, as they are in OMVs, the composition has unexpectedly high immunogenicity. Without being bound by theory it is thought that antigens presented in an OMV are correctly folded and furthermore minor membrane antigens, which are present in OMVs, will be able to further contribute to the protective effect.

The MenB strains may further comprise one, more, or all of the PorA variable region 1 (VR1) variants having the sequences set out in SEQ ID NOs: 2-5. The epitopes included in these Variable Regions have also been shown to contribute to the protective effect.

The MenB strains may also comprise PorA VR1 variant having the sequence set out in SEQ ID NO 1 [21].

Each of the different MenB strains in the composition may express exactly one PorA VR1, one PorA VR2 and one FrpB VR. When MenB strains express exactly one of each PorA VR1, PorA VR2 and FrpB VR, this avoids the problem of immunodominance of any epitope in such variable region over other epitopes expressed in such variable region by the same strain, which has previously been observed in other OMV vaccines. Instead, in the present vaccine preferably single PorA VR1, PorA VR2 and FrpB VR variants are expressed in each of the different MenB strains from which the OMVs were prepared, i.e. a single strain preferably expresses a single PorA VR1, a single PorA VR2 and a single FrpB VR, and the vaccine comprises OMVs from at least six of such single strains.

In certain embodiments, the at least six MenB strains may comprise

(i) a strain having PorA the VR2 P1.4 sequence set out in SEQ ID NO: 6 and FrpB VR F1-5 sequence set out in SEQ ID NO: 12, and/or

(ii) a strain having PorA VR2 P1.14 sequence set out in SEQ ID NO: 7 and FrpB VR F5-5 sequence set out in SEQ ID NO: 13 and/or

(iii) a strain having PorA VR2 P1.15 sequence set out in SEQ ID NO: 8 and FrpB VR F5-1 sequence set out in SEQ ID NO: 14 and/or

(iv) a strain having PorA VR2 P1.16 sequence set out in SEQ ID NO: 9 and FrpB VR F3-3 sequence set out in SEQ ID NO: 15 and/or

(v) a strain having PorA VR2 P1.9 sequence set out in SEQ ID NO: 10 and FrpB VR F5-12 sequence set out in SEQ ID NO: 16, and/or

(vi) a strain having PorA VR2 P1.2 sequence set out in SEQ ID NO: 11 and FrpB VR F4-1 sequence set out in SEQ ID NO: 17.

In certain preferred embodiments, the composition comprises OMVs from the six strains listed in (i) to (vi). MenB strains having this combination of variable region variants provides the optimal combination of strains which are most commonly observed in patients suffering from invasive meningococcal disease and provides maximum coverage of the PorA variable region 2 (VR2) (FIG. 1) and the FrpB variable region (VR) in loop 5 of fully typed MenB strains.

In certain embodiments, the at least six MenB strains may comprise:

(vii) a strain having PorA VR1 P1.7-2 (SEQ ID NO: 1); PorA VR2 P1.4 (SEQ ID NO: 6) and FrpB VR F1-5 (SEQ ID NO: 12) and/or

(viii) a strain having PorA VR1 P1.22 (SEQ ID NO: 2); PorA VR2 P1.14 (SEQ ID NO: 7) and FrpB VR F5-5 (SEQ ID NO: 13) and/or

(ix) a strain having PorA VR1 P1.19 (SEQ ID NO: 3); PorA VR2 P1.15 (SEQ ID NO: 8) and FrpB VR F5-1 (SEQ ID NO: 14) and/or

(x) a strain having PorA VR1 P1.7 (SEQ ID NO: 4); PorA VR2 P1.16 (SEQ ID NO: 9) and FrpB VR F3-3 (SEQ ID NO: 15) and/or

(xi) a strain having PorA VR1 P1.22 (SEQ ID NO: 2); PorA VR2 P1.9 (SEQ ID NO: 10)and FrpB VR F5-12 (SEQ ID NO: 16), and/or.

(xii) a strain having PorA VR1 P1.5 (SEQ ID NO: 5); PorA VR2 P1.2 (SEQ ID NO: 11) and FrpB VR F4-1 (SEQ ID NO: 17).

Preferably the composition comprises OMVs from all the six strains listed in (vii) to (xii). The immunization with the PorA/FrpB repertoires of the strains listed in (vii) to (xii) would reach ˜89% coverage in the 3553 MenB typed strains submitted to the combined national databases of The Netherlands, UK, and Poland (see ‘The National Databases’). The same PorA/FrpB repertoires would reach ˜84% coverage in the 4293 strain panel MenB typed strains submitted to the to the international PubMLST database.

The same PorA/FrpB combination would also reach ˜87% coverage of the 3873 type MenBCWY strains from The Netherlands and UK (see ‘The National Databases’), and would also reach ˜80% coverage of the 6656 typed MenBCWY strains submitted to the international PubMLST database.

The same PorA/FrpB combination would also reach ˜85% coverage of the 142 type MenW strains from The Netherlands and UK (see ‘The National Databases’), and would also reach ˜91% coverage of the 690 typed MenW strains submitted to the international PubMLST database.

The MenB strains used for preparing the OMVs of the invention may in preferred embodiments further comprise IpxL1 and rmpM mutations. Mutations in IpxL1 lead to lower LOS reactogenicity of outer membrane vesicles, while rmpM mutations lead to enhanced release of outer membrane vesicles.

The MenB strains used for preparing the OMVs of the invention may in preferred embodiments further comprise siaD and galE mutations. The siaD and galE deletions cause the absence of expression of the MenB capsule and alteration in the LOS lacto-N-neotetraose structure, respectively.

The ratio of PorA to FrpB proteins in the composition may be between 3:1 to 1:3, preferably between 2:1 and 1:2, more preferably between 1.5:1 and 1:1.5.

The invention also provides a vaccine comprising the composition as described herein.

The vaccine composition may in certain embodiments further comprise an adjuvant, optionally selected from an oil in water emulsion, liposome, saponin, lipopolysaccharide or aluminium salt. Other suitable adjuvants are known in the art.

A vaccine comprising the composition disclosed herein may be used in a method of treating or preventing infection by Neisseria bacteria in a subject, in particular infection by Neisseria meningitidis serogroup B (MenB) bacteria. The vaccine of the invention may also be effective against invasive meningococcal strains expressing A, C, W and Y capsules. The vaccine may be used in the prevention of bacterial meningitis in a subject, for example by immunization of the subject with an effective amount of the vaccine composition.

In a further aspect the invention provides a composition as described herein, for use in the manufacture of a medicament for the treatment or prevention of meningitis in a subject.

In a further aspect, the invention provides a method for producing a Neisseria meningitidis B (MenB) outer membrane vesicle composition, comprising growing at least six different MenB strains and isolating the outer membrane vesicles produced by said strains, wherein the at least six MenB strains together include the PorA VR2 P1.4, P1.9, P1.14, P1.15, P1.16 and P1.2, and FrpB VR F1-5, F5-1, F5-5, F5-12, F3-3, and F4-1. The growing step is typically done in a separate culture for each of the six strains. When the MenB strains are grown separately then the isolated OMVs will be mixed together to obtain the OMV composition.

The chemically defined medium preferably has a low iron content to induce FrpB protein expression levels that are close to those of the PorA protein expression levels. The effective concentration depends on the iron source that is used, e.g 0-22 μM, preferably 5-20 μM, for Iron (III) chloride. Optionally, an iron-chelating agent, such as deferoxamine mesylate (desferal) or ethylenediamine-N,N′-bis(2-hydroxyphenyl)acetic acid (EDDHA) is added to the medium. Preferably, the iron-chelating agent is added during growth of the MenB strains. In particular, the iron chelating agent may be added during exponential growth phase, preferably early exponential growth phase, of the MenB strains. In case of using the chelator, the effective iron concent of the medium before addition of the chelator can be increased above levels that are required to induce FrpB protein expression.

The chemically defined medium may in certain embodiments be buffered to a pH of 6.6-7.6, optionally about pH 7.2. The buffer may for instance be set to the desired pH with sodium hydroxide.

The MenB outer membrane vesicle composition may be any composition described herein.

### SUMMARY OF THE FIGURES

The invention will now be described by way of example with reference to the following figures:

FIG. 1 shows a PorA topology model with the variable regions (VR) 1 and 2 depicted on loops I and IV.

FIG. 2 shows an FrpB topology model with the variable region (VR) depicted on L5.

FIG. 3 shows a table showing the six MenB strains, together with their PorA VR1, VR2 and FrpB VR sequences which are predicted to provide the broad coverage.

FIG. 4. Chemically defined medium can be employed for the production of native OMVs (nOMVs) with equal FrpB and PorA protein content. nOMV were isolated from a N. meningitidis H44/76 RLG mutant strain grown in a shaker flask with 150 ml medium supplemented with 300 μM FeCl₃ (lane 1), in a 5 L bioreactor with 3 L medium supplemented with 12 μM FeCl₃ set at pH 7.2±0.05 (lane 2), or in a shaker flask with 150 ml medium supplemented with 16 μM FeCl₃ to which 50 μM desferal was added to at early-log phase (lane 3). nOMV protein profiles were obtained by SDS-PAGE. The position of the major nOMV proteins PorA, PorB and FrpB is indicated with arrows on the right side of the gel, the positions of the molecular weight marker is indicated on the left side of the gel. Porin B (PorB) is one of the most abundant proteins of the outer membrane and it has been found to act as adjuvans, binding TLR2 and activating dendritic cells [33, 34].

FIG. 5: SBA titres achieved in mice after immunization with nOMVs from the prototype H44/76-RLG strain containing higher or low FrpB expression. Points show data from individual mice, tested against isogenic mutants of H44/76. nOMVs were isolated from growth in shaker flasks.

FIG. 6: Activation of human TLR4 in HEK-293 cells by OMV formulations. X-axis shows the concentration of OMVs by total protein used to stimulate the cells. Y-axis shows the colorimetric readout by absorbance. nOMVs were isolated from growth in 60 L fermentors with complete extraction and purification method.

FIG. 7: Activation of human TLR2 in HEK-293 cells by OMV formulations. X-axis shows the concentration of OMVs by total protein used to stimulate the cells. Y-axis shows the colorimetric readout by absorbance. nOMVs were isolated from growth in 60 L fermentors with complete extraction and purification method.

FIG. 8: Activation of human TLR4 in HEK-293 cells by a hexavalent nOMV or licensed vaccines. X-axis shows the concentration used to stimulate the cells (1x human dose of nOMVs used here=300 μg total protein). Y-axis shows the colorimetric readout by absorbance.

FIG. 9: Activation of human TLR2 in HEK-293 cells by a hexavalent nOMV or licensed vaccines. X-axis shows the concentration used to stimulate the cells (1x human dose of nOMVs used here=300μg total protein). Y-axis shows the colorimetric readout by absorbance.

FIG. 10: Expression of inflammatory cytokine IL-6 by human PBMCs after stimulation with OMV formulations. X-axis shows the concentration of OMVs by total protein used to stimulate the cells. Y-axis shows the concentration of IL-6 measured by ELISA after 16 hours. nOMVs were isolated from growth in 60L fermentors with complete extraction and purification method

FIG. 11: Expression of inflammatory cytokine IL-1βby human whole blood (adult or cord blood) after stimulation with media only (RPMI); H44/76-RLG nOMVs (without adjuvant); H44/76-RG dOMVs (without adjuvant); H44/76-RLG dOMVs (without adjuvant); or licensed pediatric vaccines: Bexsero® (rMenB+dOMV), Prevnar 13® (conjugate vaccine covering 13 pneumococcal serotypes), EasyFive® (DTwP, HepB, HiB), PedvaxHib® (HiB-OMP). The X-axis shows the substance used to stimulate the whole blood (N=2, all stimulants at 1/10 human dose). The Y-axis shows the concentration of IL-1βmeasured by ELISA. nOMVs were isolated from growth in 60 L fermentors with complete extraction and purification method.

FIG. 12: Expression of inflammatory cytokine TNFa by human whole blood (adult or cord blood) after stimulation with media only (RPMI); H44/76-RLG nOMVs (without adjuvant); H44/76-RG dOMVs (without adjuvant); H44/76-RLG dOMVs (without adjuvant); or licensed pediatric vaccines: Bexsero® (rMenB +dOMV), Prevnar 13® (conjugate vaccine covering 13 pneumococcal serotypes), EasyFive® (DTwP, HepB, HiB), PedvaxHib® (HiB-OMP). The X-axis shows the substance used to stimulate the whole blood (N =2, all stimulants at 1/10 human dose). The Y-axis shows the concentration of TNFa measured by ELISA. nOMVs were isolated from growth in 60L fermentors with complete extraction and purification method.

FIG. 13: Expression of inflammatory cytokines IL-1β, IL-6 and TNFa by human PBMCs after stimulation with a hexavalent nOMV formulation or licensed vaccines. X-axis shows the concentration used to stimulate the cells (1×human dose of nOMVs used here=30μg total protein). Y-axis shows the concentration of each cytokine measured by ELISA.

FIG. 14: SBA titers against a P1.7-2,4;F1-5;cc41/44 target strain after immunization of mice with 2 doses each of a hexavalent nOMV formulation (30 μg total protein/dose), a hexavalent dOMV formulation (30 μg total protein/dose with Al(OH)₃ adjuvant), Bexsero® (1/5^{th} human dose) or a buffer control. nOMVs and dOMVs were tested in two separate studies, with Bexsero used as a comparator in each study. The graph shows titers of individual mice with the geometric mean titer for each group.

FIG. 15: SBA titers against a panel of target strains after immunization of mice with 2 doses each of a hexavalent nOMV formulation (30 μg total protein/dose), Bexsero® (1/5^{th} human dose) or a buffer control. The graph shows titers of individual mice with the geometric mean titer for each group.

### DETAILED DESCRIPTION

The present disclosure provides immunogenic MenB compositions, including vaccine compositions, comprising particular PorA and FrpB variants. Methods of producing such compositions are also provided.

### Protein Variable Regions and Epitopes

The PorA and FrpB (also called FetA) outer membrane proteins (OMPs) of N. meningitidis have been previously characterised and epitopes have been described within the Variable Regions (VRs) of these proteins [4-6].

Variable regions are generally assumed to coincide with single epitopes, even though not all described VRs have been demonstrated to react with a monoclonal antibody.

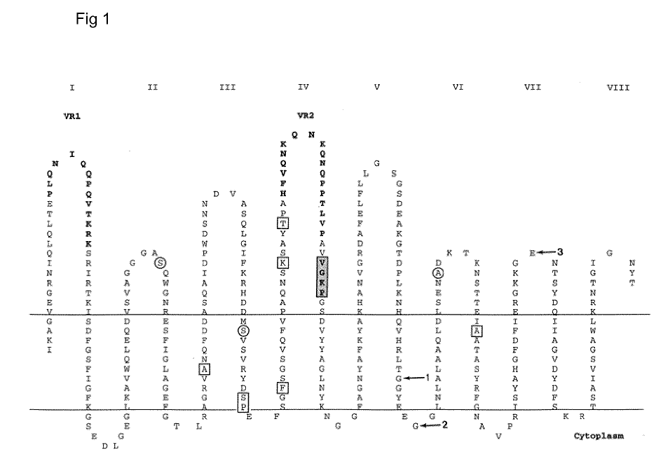

Most of the variability in the PorA protein occurs in two of the eight putative exposed surface loops. These variable loops, (I and IV) have been designated Variable Region 1 and Variable Region 2 (VR1 and VR2) respectively. Hundreds of variants of these variable regions have been described, see for example [5 and 6]. A PorA topology model with VR1 and VR2 indicated is shown in FIG. 1.

Most variability in FrpB has been found to occur at a single variable region (VR), which includes bactericidal epitopes [18, 20]. Hundreds of variants of this region have been described, see for example [4].

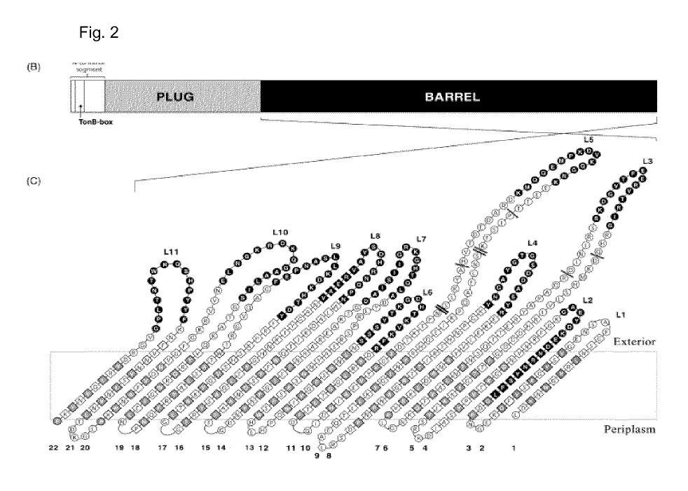

An FrpB topology model is shown in FIG. 2. The variable region is located in the extracellular loop 5 (L5). This variable region is not involved in the suggested iron transport function of FrpB, but is thought to shield the conserved functional parts of the transporter from the immune recognition [35].

PorA VR1 and VR2 variants are generally indicated with numbers, as, for example: P1.7-2,4 where 7-2 is the PorA VR1 and 4 is the PorA VR2. Where only one VR is specified, it will be indicated whether this is the VR1 or VR2. As there is only one variable region in FrpB there is no need to specify this type of detail.

Variable regions designated with a single number (e.g. 4) are demonstrated epitopes recognised by given monoclonal antibodies. The sub-variants (e.g. 7-2, or 10-15), which are recognizable by a dash after the number of the main family, are often not recognized by the mAb which recognized the head of the family (e.g. the sub-variant 7-2 may not be recognised by the mAb which recognised 1.7). Those sub-variants can differ by as little as one amino acid from the head of the family. For convenience, the term ‘variant’ will be used to refer to demonstrated epitopes and sub variants.

The genes encoding meningococcal outer membrane proteins (OMPs) are generally quite variable, due to the strong selection pressure exerted on the regions encoding the parts of the proteins exposed to the immune system. A consequence thereof is that the immune responses raised by these proteins are mainly limited to homologous meningococcal strains.

Despite the high variability of OMPs, the present inventors have discovered that a limited sub-set of PorA and FrpB variants can provide protection against the majority of different MenB strains currently in circulation. The diversities of PorA and FrpB proteins were shown to be highly structured among hyperinvasive lineages [17]. Therefore, a vaccine including OMV extracted from strains displaying selected PorA/FrpB variant combinations can complement the lack of immune response to one of the antigens by the robust production of bactericidal antibodies in response to the second antigen.

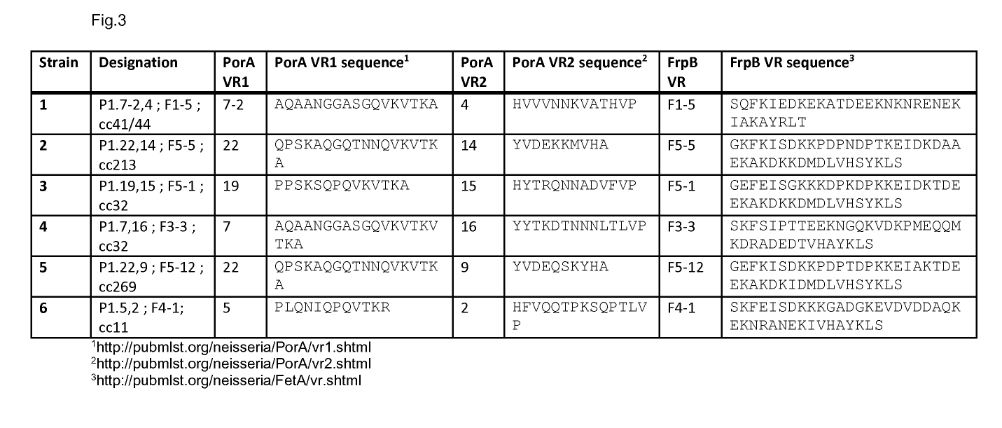

The variable regions encompassing the relevant epitopes according to the present invention are listed in the Table shown in FIG. 3. The sequences corresponding to each of these variable regions are included in the sequence listing and are referred to below.

The MenB strains described herein comprise a PorA antigen which has a VR2 selected from P1.4 (SEQ ID NO: 6); P1.9 (SEQ ID NO: 10); P1.14 (SEQ ID NO: 7); P1.15 (SEQ ID NO: 8); P1.16 (SEQ ID NO: 9) and P1.2 (SEQ ID NO: 11). These PorA VR2 variants may be found in combination with any known PorA VR1 variant, preferably any PorA VR1 variant disclosed herein.

The MenB strains described herein also comprise an FrpB VR selected from F1-5 (SEQ ID NO: 12); F5-1 (SEQ ID NO: 14); F5-5 (SEQ ID NO: 13); F5-12 (SEQ ID NO: 16); F3-3 (SEQ ID NO: 15) and F4-1 (SEQ ID NO: 17).

The PorA antigen may also have a VR1 selected from P1.7 (SEQ ID NO: 4); P1.19 (SEQ ID NO: 3); P1.7-2 (SEQ ID NO: 1); P1.22 (SEQ ID NO: 2); or P1.5, (SEQ ID NO: 5).

The MenB strains described herein may for instance comprise PorA (VR1,VR2) antigen selected from P1.7-2,4 having the sequences set out in SEQ ID NOs 1 and 6; P1.22, 14 having the sequences set out in SEQ ID NOs 2 and 7; P1.19,15 having the sequences set out in SEQ ID NOs 3 and 8; P1.7, 16 having the sequences set out in SEQ ID NOs 4 and 9; P1.22,9 having the sequences set out in SEQ ID NOs 2 and 10; and P1.5,2 having the sequences set out in SEQ ID NOs 5 and 11.

Preferably the MenB strains described herein comprise a PorA selected from P1.7-2,4, P1.22,14, P1.19,15, P1.7,16 P1.22,9, and P1.5,2 having the sequences set out above and an FrpB VR selected from F1-5 having the sequence set out in SEQ ID NO: 12, F5-1 having the sequence set out in SEQ ID NO: 14, F5-5 having the sequence set out in SEQ ID NO: 13, F5-12 having the sequence set out in SEQ ID NO: 16, F3-3 having the sequence set out in SEQ ID NO: 15 and F4-1 having the sequence set out in SEQ ID NO: 17.

The MenB strains described herein may comprise one, two, three, four, five, or six of the following strains (i) to (vi), or any combination of the PorA VR2 and FrpB VR variants thereof:

(i) a strain having the PorA VR2 P1.4 sequence set out in SEQ ID NO: 6 and FrpB F1-5 VR sequence set out in SEQ ID NO: 12,

(ii) a strain having the PorA VR2 P1.14 sequence set out in SEQ ID NO: 7 and FrpB F5-5 VR sequence set out in SEQ ID NO: 13,

(iii) a strain having the PorA VR2 P1.15 sequence set out in SEQ ID NO: 8 and FrpB F5-1 VR sequence set out in SEQ ID NO: 14,

(iv) a strain having the PorA VR2 P1.16 sequence set out in SEQ ID NO: 9 and FrpB F3-3 VR sequence set out in SEQ ID NO: 15,

(v) a strain having the PorA VR2 P1.9 sequence set out in SEQ ID NO: 10 and FrpB F5-12 VR sequence set out in SEQ ID NO: 16,

(vi) a strain having the PorA VR2 P1.2 sequence set out in SEQ ID NO: 11 and FrpB F4-1 VR sequence set out in SEQ ID NO: 17.

Preferably the composition comprises OMVs from each of the six strains listed in (i) to (vi).

The at least six MenB strains described herein may comprise one, two, three, four, five, or six of the following strains (vii) to (xii), or any combination of the PorA VR1, VR2 and FrpB VR variants thereof:

(vii) a strain having the PorA VR1 P1.7-2 sequence set out in SEQ ID NO: 1; PorA VR2 P1.4 sequence set out in SEQ ID NO: 6 and FrpB VR F1-5 sequence set out in SEQ ID NO: 12,

(viii) a strain having the PorA VR1 P1.22 sequence set out in SEQ ID NO: 2; PorA VR2 P1.14 sequence set out in SEQ ID NO: 7 and FrpB VR F5-5 sequence set out in SEQ ID NO: 13,

(ix) a strain having the PorA VR1 P1.19 sequence set out in SEQ ID NO: 3; PorA VR2 P1.15 sequence set out in SEQ ID NO: 8 and FrpB VR F5-1 sequence set out in SEQ ID NO: 14,

(x) a strain having the PorA VR1 P1.7 sequence set out in SEQ ID NO: 4; PorA VR2 P1.16 sequence set out in SEQ ID NO: 9 and FrpB VR F3-3 sequence set out in SEQ ID NO: 15,

(xi) a strain having the PorA VR1 P1.22 sequence set out in SEQ ID NO: 2; PorA VR2 P1.9 sequence set out in SEQ ID NO: 10 and FrpB VR F5-12 sequence set out in SEQ ID NO: 16,

(xii) a strain having the PorA VR1 P1.5 sequence set out in SEQ ID NO: 5; PorA VR2 P1.2 sequence set out in SEQ ID NO: 11 and FrpB VR F4-1 sequence set out in SEQ ID NO: 17.

Preferably the composition comprises OMVs from each of the six strains listed in (vii) to (xii).

### Number of MenB Strains

The composition described herein comprises outer membrane vesicles (OMVs) from at least six different MenB strains having PorA VR2 and FrpB variants as described herein.

The composition may further comprise OMVs from one or more additional MenB strains. For example the composition may comprise OMVs from at least 6, at least 7, at least 8, at least 9 or at least 10 MenB strains. The composition may comprise OMVs a total of 6, 7, 8, 9, or 10 different MenB strains or any range selected from these values (e.g. the composition may comprise OMVs from 6-10, 6-8 MenB strains etc.).

The one or more further MenB strains may include strains having a PorA VR1 variant, PorA VR2 variant and/or FrpB variant as disclosed in FIG. 3 together with one or more PorA VR1, VR2 and/or FrpB variants not disclosed in FIG. 3. For example a MenB strain may have the PorA VR1 P1.7-2 variant (which is disclosed in FIG. 3) together with a PorA VR2 variant and FrpB variant which are not disclosed in FIG. 3. It is also envisaged that the one or more further MenB strains may include PorA VR1, VR2 and FrpB variants not disclosed in FIG. 3.

The inclusion of additional MenB strains allows the composition to be tailored to specific needs (e.g. specific outbreaks) by including additional MenB strains with different variable regions to those in FIG. 3. In turn this will further increase the breadth of protection provided by the composition.

Each MenB strain preferably expresses exactly one PorA protein and exactly one FrpB protein. This is the same as that found in wild-type (i.e. not recombinant) MenB strains. Thus, each MenB strain will preferably express exactly one PorA VR1, one PorA VR2 and one FrpB VR.

It is also possible that a MenB strain is engineered to express more than one PorA protein and/or more than one FrpB protein. An advantage of using MenB strains expressing exactly one PorA protein and exactly one FrpB protein is that there is no risk for unbalanced surface exposure or immunogenicity between different PorA or FrpB variant proteins in a single strain, and hence no risk for exacerbating uneven immune responses against variants of the same protein.

### Iron-Regulated Proteins

Each of the MenB strains expresses FrpB. Unlike PorA, which is expressed in most growth conditions, FrpB is poorly expressed in most growth conditions.

FrpB is an iron-inducible (also called iron-regulated) protein. The iron-regulated proteins are a relatively well-studied class of outer membrane proteins which are expressed during iron-limited growth conditions. These include receptors that are involved in the uptake of iron from sources such as siderophores, transferrin and haemoglobin. The concentration of free soluble iron in the host tissues is insufficient to support microbial growth. Therefore the ability to utilize iron from available sources such as these is believed to play an important role in colonization dissemination of meningococci in the human body.

High level FrpB expression has previously been reached in complex liquid medium to which an iron-chelator was added [22]. Alternatively, high level FrpB expression can be achieved in chemically defined growth medium. Alternatively, high FrpB expression can be accomplished by introducing genetic modifications in the bacterial genome, which could have a profound influence on the growth characteristics and antigenic profiles of bacterium [19]. Over-expression of FrpB has previously shown to be difficult without using an inducible plasmid [18], but stability of a self-replicating high-copy expression vector in a vaccine production strain could prove difficult.

In extremely iron-limited conditions the bacterial strains may struggle to grow and the biomass of a bacterial culture may be reduced. Therefore it is generally preferred to achieve a balance between reducing the iron content of a growth medium to increase expression of iron-regulated proteins, and maintaining a good yield of bacteria from a growth culture.

Growing MenB strains in the chemically defined media described herein has been shown to induce FrpB expression, while at the same time supporting bacterial growth and good PorA expression.

In certain preferred embodiments FrpB makes up at least 5, 6, 7, 8, 9, 10, 11, 12, 13, 14, 15, 16, 17, 18, 19 or 20% of the total protein expressed by the MenB strains.

In certain preferred embodiments, PorA makes up at least 15, 16, 17, 18, 19, 20, 21, 22, 23, 24 or 25% of the total protein expressed by the MenB strains.

The amount of a given protein compared to the total protein can for instance be determined by densitometric analysis of bands on protein gels, or by other known methods.

Increasing the amount of PorA and FrpB proteins expressed by the MenB strains leads to a corresponding increase in the amount of these proteins (and so the immunogenic PorA and FrpB variants) in the OMVs.

In preferred embodiments, the ratio of PorA to FrpB proteins in the composition may be between 3:1 to 1:3, preferably between 2:1 and 1:2, more preferably between 1.5:1 and 1:1.5. The ratio is calculated by measuring the total PorA and FrpB protein by SDS-PAGE, followed by total protein staining with ‘Coomassie’ triphenylmethane dyes and quantification of the 40-44 kD PorA bands and the ˜70 kDa FrpB band of the OMVs from the different MenB strains included in the composition (see e.g. [31]), and calculating the ratio between these proteins.

The ratio of PorA to FrpB proteins in the composition in the composition can be changed by inducing FrpB expression (e.g. by iron-limitation). As PorA is not induced by iron-limitation then increasing FrpB expression in this way will decrease the ratio of PorA to FrpB.

FrpB is a major iron-regulated outer membrane protein. However, many other examples of minor iron-regulated proteins have been described and include LbpA, LbpB, TbpA, TbpB, LbpA, and HmbR.

A number of these iron-regulated proteins have been shown to be immunogenic, although this is generally against a limited number of MenB strains. For example, LbpB (lactoferrin-binding protein B) has been shown to be a target for bactericidal antibodies, but has limited cross-reactivity [14]. Growing MenB strains in the chemically defined media described herein, which has been shown to optimize FrpB expression, is also expected to increase expression of minor iron-regulated proteins which may enhance the overall protective effect of the composition.

### OMVs

The composition comprises outer membrane vesicles (OMVs) from different MenB strains. Outer membrane vesicles are released from the N. meningitidis outer membrane and contain outer membrane proteins (OMP) and lipooligosaccharides (LOS) in their natural conformation and membrane environment.

The OMVs are prepared by inducing OMV blebbing and isolating the OMVs from the N. meningitidis bacterial suspension and growth medium. Methods for preparing OMVs are well known in the art [11]. In traditional OMV vaccine preparation, a concentrated bacterial biomass suspension is treated with detergent (dOMV). In this process, commonly used detergent deoxycholate (DOC) induces vesicle formation from the bacterial outer membrane along with stripping of (toxic) LPS, phospholipids, and (immunogenic) lipoproteins. The removal of these membrane constituents has shown to result in OMV aggregates with significant size heterogeneity and moreover reduced cross protection of minor antigens.

Preferably, the OMVs are prepared without detergent extraction. Methods for preparing OMVs that do not require a detergent extraction step have been described [11]. Preparing OMVs by a detergent-free process preserves the native vesicle structure which results in an homogeneous more mono dispersed OMV suspension with retained membrane immunogenic profile. Two detergent free OMV recovery processes have been described. The native (nOMV) process is similar to the dOMV process except for the replacement of amphipathic detergent for a metal chelating agent which promotes vesicle formation by removal of membrane stabilizing magnesium ions [31]. In the spontaneous OMV (sOMV) process, vesicle formation is induced during the bacterial growth by metabolic conditioning of the cells. The vesicles are retrieved directly from the supernatant of the fermentation broth by centrifugation method [11] or filtration method [32]. Irrespective of nOMV primary recovery method, the OMV purification protocol typically contains one or more size separation method to concentrate and purify the OMVs from host cell proteins, lipids, low molecular weight nucleic acids, and non bound LPS. DNA size can be reduced by use of an endonuclease step. An example of a complete nOMV vaccine manufacturing protocol is for instance described in [31]. The native (nOMV) process is preferred for use in relation to the present compositions and methods. Thus, preferably the MenB OMVs included in the composition are nOMVs.

The OMVs may be extracted from the different MenB strains separately, and then combined into a single composition.

Preferably the MenB strains are cultured under conditions which maximise the expression of outer membrane proteins in the OMVs as described herein.

### Mutations

The MenB strains may be genetically engineered to include mutations in one or more of the rmpM, IpxL1, siaD-galE genes. Preferably all of the MenB strains in the composition have mutations in all of the rmpM, IpxL1, siaD-galE genes. Strains carrying these mutations may be referred to as ‘RLG’ strains.

The rmpM, IpxL1 and/or siaD-galE mutations are knockout (KO) mutations. A knockout mutation of a target gene is one which alters the sequence of the gene such that the gene expression is undetectable or insignificant, and/or the gene product does not function or can be considered not significantly functional. For example a knockout of the IpxL1 gene means that the function of the gene has been significantly decreased so that the expression of the gene is not detectable or is only present at insignificant levels and/or a biological activity of the gene product is significantly reduced relative to prior to the modification or is not detectable.

Methods for mutating these genes, and producing ‘RLG’ strains, have been described previously in Neisseria bacteria. For example, rmpM gene (e.g. [10]) (i.e. R mutation), IpxL1 gene (i.e. L mutation) (e.g. [7]), and siaD-galE locus (i.e. G mutation) (e.g. [8], [9]).

KO-mutations in the rmpM gene leads to increased release of OMVs [11].

KO-mutations in the IpxL1 gene leads to expression of the penta-acylated lipid A form of lipooligosaccharide (LOS), instead of hexa-acylated lipid A as present in wild-type LOS. The reduced reactogenicity of the penta-acylated LOS avoids the need for LOS removal from the OMV [7].

KO mutations of the siaD -and of the galE genes lead to the absence of capsule B capsular polysaccharide, and to the production of truncated oligosaccharide, respectively. These modifications make the vaccine strain non-pathogenic and devoid of potential for cross-reactivity with human antigens [9].

A genomic modification to include in the vaccine strains, as an alternative to rmpM mutations, could be a KO-mutation in the gene coding for GNA33 protein. This protein is a lytic transglycosylase involved in the maintainance of the bacterial cellular structure. Like rmpM mutants, meningococcal strains which have been knocked-out for the gna33 gene spontaneously release increased amounts of OMV vesicles, without need of any further chemical/physical treatment [29].

KO-mutations in IpxL2 can be used as alternatives, or in addition, to IpxL1 mutations (even though the benefits of inactivating IpxL2 instead of IpxL1, or in addition to IpxL1 have not been proven) [7].

It is to be understood that although the MenB strains may include mutations in the rmpM, IpxL1, siaD-galE genes, and are therefore not native (or wild-type) strains, they retain the native (naturally-occurring) porA and frpB sequences as they are typically found in Neisseria meningitidis bacteria. Preferably this also includes native porA and frpB promoter sequences. In certain preferred embodiments, the MenB strains, other than having rmpM, IpxL1 and/or siaD-galE mutations, will further be the same as the naturally occurring MenB strains.

In some embodiments, the MenB strains may be genetically engineered to increase FrpB expression relative to a wildtype strain. This can be achieved by, for example, the replacement of the native frpB promoter for a stronger promoter (e.g. [23]), placing the frpB gene on different genetic loci in bacterial chromosome (e.g. [36], [19]), or on a plasmid (e.g. [18]), cosmid or other mobile element. Examples of alternative promoters that can be placed in front of the frpB gene in all genetically engineered strains include but are not limited to the frpB promoters from a different Neisseria meningitidis strain, a genetically modified native frpB promoter [19], promoters from other Neisseria meningitidis genes that have high protein expression levels, such as porA, porB, rmpM, and opa, promoters from other species or artificial promoters that have been engineered to be active in Neisseria meningitidis.

In addition, in certain embodiments some or all of the MenB strains are further engineered to overexpress factor H-binding protein (fHpb; also been known as protein 741, NMB 1870, GNA1870, P2086, LP2086 or ORF2086) relative to a wild type strain. Overexpressing fHpb in the MenB strains will increase the amount of fHpb in the OMVs from these strains. The expression of fHpb may be increased in one, two, three, four, five, six, or more of the MenB strains used to prepare the composition. By further increasing the expression of the proven immunogenic fHbp protein [30] in some or all of the strains used for preparing the OMVs of the compositions of the invention, the immune response to the vaccine compositions could be enhanced and/or broadened even further. Factor H-binding protein variants have been categorised into two families A and B. In certain aspects, at least one representative fHbp member of family A and one of family B can be overexpressed in the MenB strains, leading to a corresponding increase in fHpb in the OMV from these strains, see [30]. In certain aspects these at least two fHbps include variant A05 and B01 [39, 40]. Further variants of fHbp could also be included. In particular, the OMVs in the composition of the invention provide the possibility to have overexpression and strong antigenic presentation in the form of an OMV for at least six variants (one per MenB strain) of the fHbp protein. Overexpression of fHbp in the strains can be done by routine genetic engineering, e.g. by placing the gene encoding fHpb under control of heterologous promoters, e.g. iron-regulated promoters, or other methods as described above for FrpB.

### Vaccine Compositions

The present invention also provides vaccine composition comprising a therapeutically effective amount of a composition described herein.

The composition may be prophylactic (i.e. to prevent infection with Neisseria bacteria).

A disease caused by Neisseria bacteria encompasses any clinical symptom or combination of symptoms that are present in an infection with Neisseria meningitidis bacteria. These symptoms include but are not limited to: colonization of the upper respiratory tract by a pathogenic strain of Neisseria meningitides (e.g. mucosa of the tonsils and nasopharynx), penetration of the bacteria into the mucosa and the submucosal vascular bed, septicemia, septic shock, inflammation, haemorrhagic skin lesions, activation of fibrinolysis and of blood coagulation, organ dysfunction (e.g. kidney, lung, and cardiac failure), adrenal hemorrhaging and muscular infarction, capillary leakage, edema, peripheral limb ischaemia, respiratory distress syndrome, pericarditis and meningitis.

The composition may be for human usage in human medicine. Preferably the composition is for administration to a subject. Preferably the subject is human.

The compositions described herein may be formulated with a pharmaceutically acceptable carrier, excipient, buffer, stabilizer or diluent or other materials well known to those skilled in the art. Suitable pharmaceutically acceptable carriers, excipients or diluents are described, for example, in (Remington's Pharmaceutical Sciences, 18th edition, A. R. Gennaro, Ed., Mack Publishing Company [1990]; Pharmaceutical Formulation Development of Peptides and Proteins, S. Frokjaer and L. Hovgaard, Eds., Taylor & Francis [2000]; and Handbook of Pharmaceutical Excipients, 3rd edition, A. Kibbe, Ed., Pharmaceutical Press [2000]). The precise nature of the carrier or other material will depend on the route of administration.

The compositions described herein may further comprise an adjuvant. The adjuvant may for instance be selected from an oil in water emulsion, liposome, saponin, lipopolysaccharide or aluminium salt. Suitable adjuvants are well known in the art.

A ‘therapeutically effective amount’ means a sufficient amount of a composition to show benefit to a subject, including, but not limited to, inducing/increasing an immune response against Neisseria bacteria in a subject, reducing the severity or duration of meningitis disease in a subject. The actual amount administered, and rate and time-course of administration, will depend on the nature and severity of what is being treated. Prescription of treatment, e.g. decisions on dosage etc, is within the responsibility of general practitioners and other medical doctors and may depend on the severity of the symptoms and/or progression of a disease being treated.

An immune response is induced or increased if these is a detectable difference in an immunological response indicator measured before and after administration of a particular composition. Immune response indicators include but are not limited to: antibody titer or specificity, as detected by an assay such as enzyme-linked immunoassay (ELISA), bactericidal assay, flow cytometry, immunoprecipitation, Ouchter-Lowny immunodiffusion; binding detection assays of, for example, spot, Western blot or antigen arrays; cytotoxicity assays, etc.

The present invention also provides methods for immunizing a subject against Neisseria meningitides infection, the method comprising administering to said subject a vaccine composition as described herein.

### Administration

Compositions as described herein, including vaccine compositions, may be administered via any suitable route, for example, parenteral (in injectable form), mucosal, e.g. intranasal or oral (for example as a spray, tablet or capsule), or topical (for example as a cream or lotion).

Some preferred routes of parenteral administration for the vaccines of the invention are intramuscular, subcutaneous, or intradermal injection, intramuscularly being particularly preferred. A preferred mucosal route of administration for the vaccines of the invention is intranasal administration. An alternative route of administration may be via the skin. For infants, intramuscular administration is particularly preferred. For adolescents, intramuscular is possible, but alternative delivery routes such as intranasal administration or administration via the skin are also possible.

Other suitable routes of administration are well known in the art.

The composition may be formulated in a form which is appropriate for the intended mode of administration. For example, as a powder, spray, tablet, solution, or suspension, optionally together with suitable carriers, excipients or diluents (or a combination thereof). For parental administration the composition may be in the form of a sterile aqueous solution and may optionally contain other substances, for example salts or buffers. Those of skill in the art are well able to prepare suitable solutions using, for example, isotonic vehicles such as Sodium Chloride Injection, Ringer's Injection, Lactated Ringer's Injection.

Preservatives, stabilisers, buffers, antioxidants and/or other additives may be included, as required including buffers such as phosphate, citrate and other organic acids; antioxidants, such as ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3′-pentanol; and m-cresol); low molecular weight polypeptides; proteins, such as serum albumin, gelatin or immunoglobulins; hydrophilic polymers, such as polyvinylpyrrolidone; amino acids, such as glycine, glutamine, asparagines, histidine, arginine, or lysine; monosaccharides, disaccharides and other carbohydrates including glucose, mannose or dextrins; chelating agents, such as EDTA; sugars, such as sucrose, mannitol, lactose, trehalose or sorbitol; salt-forming counter-ions, such as sodium; metal complexes (e.g. Zn-protein complexes); and/or non-ionic surfactants, such as TWEEN™, PLURONICS™ or polyethylene glycol (PEG).

A composition described herein may be administered alone or in combination with other treatments, either simultaneously or sequentially.

Administration may be repeated at daily, twice-weekly, weekly or monthly intervals. The treatment schedule for an individual subject may be dependent on factors such as the route of administration and the severity of the condition being treated.

The composition may be administered in one or more doses which may be followed by one or more further ‘booster’ doses which are administered days, weeks or years later. For example, when administering the composition to children, a first dose may be given at 11-12 years of age and a booster dose at 16 years of age. For adolescents who receive the first dose at 13-15 years of age, a booster dose may be given at 16-18 years of age. The injections may contain the same dose of active ingredient or may contain different doses. Preferably the dose will be administered by injection.

The composition may be administered to children, adolescents or adults. ‘Children’ includes infants who are generally those up to 2 years of age. Infants will generally be administered two doses with a third ‘booster’ dose administered in the second year of life.

The specific dose level and frequency of dosage for any particular patient may be varied and will depend upon a variety of factors including the age, body weight, general health, sex, diet, mode of administration of the individual undergoing treatment. Typically a suitable dosage may be determined by a physician.

The composition may be administered as a dose from 0.00001 μg/Kg body weight to body weight to 5 mg/Kg body weight, preferably 0.0001 μg/Kg to 5mg/Kg, preferably 0.001 μg/Kg to 1mg/Kg, preferably 0.01 μg/Kg to 500 μg/Kg, preferably 0.02 μg/Kg to 300 μg/Kg body weight.

A composition described herein may be provided in the form of a kit, e.g. sealed in a suitable container which protects its contents from the external environment. Such a kit may include instructions for use.

### Methods for Growing MenB Strains.

As both PorA and FrpB variants have been identified as being important in providing an immunogenic response against a broad range of MenB strains, it is desirable to optimize the expression of these proteins.

The present inventors give examples of growth conditions in order to increase expression of PorA and FrpB in the outer membrane vesicles produced by the bacterial strain. Advantageously, these growth conditions also increase the expression of other iron-regulated proteins, in addition to FrpB, which are expected to further increase the immunogenicity of the composition (e.g. [23]).

Accordingly, the present invention provides a method for producing a MenB outer membrane vesicle composition, which may be a vaccine composition, comprising growing at least six MenB strains as disclosed herein and isolating the outer membrane vesicles produced by the strains to obtain the OMV composition.

Each of the MenB strains will typically be grown in a separate culture. When the MenB strains are grown separately then the isolated OMVs will be mixed together to obtain the OMV composition.

Media suitable for supporting the growth of MenB are well known in the art and include, chemically defined and chemically undefined media. A chemically undefined medium is one which has some complex ingredients, such as yeast extract, which consist of a mixture of multiple chemical species in unknown proportions. Suitable chemically undefined media are known in the art and include, for example Frantz complete medium. A chemically defined medium is one which contains a number of defined nutrients and components to support growth of N. meningitidis bacteria. Suitable chemically defined media are known in the art, for example [13, 14, 15]. Preferably the MenB strains are grown in a chemically defined medium.

Preferably, the medium has low iron (III) content that is inducing the expression of iron-regulated proteins. Such conditions are sometimes referred to as ‘Iron limiting’ or ‘growth limiting’ conditions, have been described before to increase the expression of iron-regulated proteins such as FrpB, and are thus known to the skilled person (especially in complex media using iron chelators, e.g. [18, 22], but such chelators could also be used in chemically defined media, and/or the concentration of iron can be easily manipulated by changing the amount of iron source in such defined media). A ‘low’ iron content is generally around 22 μM or less. The iron content of the medium may therefore be between 0 and 22 μM, preferably 5-20 μM. For example, the FeCl₃ content of a chemically defined medium may be between 5 and 22 μM, e.g. 12 μM. Other sources of iron are possible, and the optimal iron (III) concentration for expression can be different for other iron (III) sources.

The method may further comprise adding an iron chelator (iron chelating agent) to the medium. An iron chelating agent may be added to the medium to reduce the amount of available iron and so increase expression of iron-regulated proteins. Suitable iron chelators for use in the method are known in the art and include, for example, desferal (desferrioxamine), or EDDHA (ethylenediamine-N,N′-bis(2-hydroxyphenylacetic acid).

The iron chelator may be added to the medium during growth of the bacterial culture, for example, during the exponential growth phase of the bacterial culture. Preferably the iron chelator is added during early exponential growth phase. Early exponential growth is typically achieved between 0 and 4 hours after inoculating the bacterial culture, and is recognized by an accelerated increase in the culture's optical density after an initial lag phase of poor bacterial growth.

The pH of the medium may be kept within a particular range during growth by using a buffer, base and/or acid well known to the art. In certain embodiments, the pH range is kept relatively narrow. For example, keeping the pH of the medium constant at pH 7.2±0.05 with sodium hydroxide and phosphoric acid results in high levels of FrpB expression.

The OMVs are extracted from the MenB strains, preferably when the culture has reached the late-exponential or stationary growth phase. External stress stimuli, such as cysteine depletion, may optionally be used to enhance OMV release [41].

Methods for extracting OMVs from MenB bacteria are known in the art. Preferably the OMVs are extracted without detergent as described herein. A suitable production process is described in [31].

Each and every compatible combination of the embodiments described above is explicitly disclosed herein, as if each and every combination was individually and explicitly recited.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Any undefined terms have the meanings recognized in the art.

### EXPERIMENTAL

Certain aspects and embodiments of the invention will now be illustrated by way of example and with reference to the figures described above.

### Materials and Methods

MenB H44/76 ‘RLG’ mutant was genetically engineered to contain the functional disruption of rmpM gene (i.e. R mutation), IpxL1 gene (i.e. L mutation) and siaD-galE locus (i.e. G mutation) as previously described [7-10].

Protein expression levels were determined by densitometric analysis of ‘Coomassie’-stained SDS-PAGE gels. The percentage of antigen relates to the total mass of protein in the sample. Densitometry of SDS protein bands was carried out by routine methods, e.g. as described in [31].

Mass spectrometry (to identify protein bands) was carried in-gel trypsin digestion of excised proteins bands and NanoLC-MS/MS analysis of extracted peptides, such as described in [38].

Chemically defined media for the growth of Neisseria meningitidis bacteria have been previously described, for example [13 and 14]. Bacterial cultures were performed in 500 mL baffled shaker flasks with 150 mL medium or in 5 L bioreactors with 3 L medium as described (e.g. [31]).

Outer membrane vesicles that were representative for nOMV were isolated from bacterial culture cell pellets by an EDTA extraction and Ultracentrifugation according to published methods (e.g. [31]). dOMVs were also prepared from wildtype, RG or RLG strains grown under the same conditions as used for nOMV extraction or in RPMI media. dOMVs were extracted as described by [12].

Female Balb/c mice (10/group) were immunized with 2 doses each (5 μg total protein per dose or 2.5 μg total protein per dose) of H44/76-RLG nOMVs, H44/76-RG dOMVs, or Bexsero® (4CMenB Meningococcal B vaccine, Novartis Vaccines) (1/10^{th} human dose) on days 0 and 28. Terminal bleeds were taken on day 42. In a second experiment, female Balb/c mice (10/group) were immunized with 2 doses of a combination of five RLG nOMVs (12.5 μg or 25 μg total protein per dose) or Bexsero® (4CMenB Meningococcal B vaccine, Novartis Vaccines) (1/10^{th} human dose) using the same schedule. In a third study, female Balb/c mice (20/group) were immunized with 2 doses of a combination of six RLG nOMVs (30 μg total protein per dose), six wildtype dOMVs with Al(OH)₃ adjuvant (30 μg total protein per dose), or Bexsero® (4CMenB Meningococcal B vaccine, Novartis Vaccines) (1/5^{th} human dose) using the same schedule described above. Evaluation of bactericidal activity was performed with sera from individual mice as described previously [24].

HEK-293 cells expressing hTLR4 and hTLR2 (Invivogen, San Diego, USA) were grown, maintained and stimulated according to the manufacturer's instructions (see also [24]). For stimulation, cells were incubated with the vaccine samples for 24 hours. Levels of SEAP activity were measured using HEK-Blue Detection (Invivogen). nOMVs were compared to Bexsero® (Novartis) and PedvaxHIB® (Haemophilus b Meningococcal Protein Conjugate vaccine), Merck Vaccines).

Stimulation of Peripheral Blood Mononuclear Cells (PBMCs, obtained from Sanquin Blood Supply, Amsterdam, Netherlands) was performed as described previously [26]. Cytokine induction in cell culture supernatant was measured using a 10-plex Human Proinflammatory Panel 1 (V-PLEX) ELISA kit from Meso Scale Discovery (Rockville, USA). nOMVs were compared to dOMVs from the RG and RLG strains, and to Bexsero® (Novartis) and PedvaxHIB® (Haemophilus b Meningococcal Protein Conjugate vaccine, Merck Vaccines).

Whole blood assays were performed as described previously [27] with either adult blood or cord blood. Blood samples were stimulated with vaccine samples at 1/10^{th} human dose.

### Example 1. Broad MenB Strain Coverage by a Combination of PorA and FrpB Variants in OMVs According to the Invention

To determine the predicted MenB coverage of a vaccine composed of OMVs from six MenB strains together having the variable regions depicted in FIG. 3, the meningococcal Molecular Epidemiology databases available at three European National Neisseria Reference Laboratories, i.e. The Netherlands (years 2002-2012), United Kingdom (years 2010-2014) and Poland (years 2007-2011), and the international PubMLST database (years 2000-2014) were used. The three national databases were combined to yield a single database with 3553 strains, while the PubMLST database with 4293 international strains was used independently. Only fully fine-typed MenB strains were included.

As based on PorA VR1, PorA VR2 and FrpB VR typing, the bactericidal immune response against the strains depicted in FIG. 3 would reach ˜89% and 84% coverage of the 3553 strains from the national databases and the 4293 strains from the PubMLST database, respectively. In all cases PorA VR1 type P1.7-2 was excluded from the cumulative coverage calculations as this PorA VR1 type is known not to provide protection. These coverage estimates are higher compared to those reached by the combinations of six purified PorA and five purified FrpB proteins (referred to as ‘standard’ combinations in [3]) that other researchers proposed [3], [17], which would cover 85% of the isolates from the national databases and 81% of the isolates in PubMLST. For the ‘enhanced’ combination of seven purified PorA and five purified FrpB antigens that these researchers proposed [3], coverage would be 86% for the national databases and 84% for PubMLST. This means that higher or similar coverage protection would be achieved with the OMV composition of the present invention, while requiring fewer PorA antigens (one fewer PorA antigen than the ‘standard’ combination and two fewer PorA antigens than the ‘enhanced’ combination in [3]). Moreover, compared to purified recombinant proteins, the OMV vaccines of the present invention have the additional advantage of comprising naturally folded proteins in their natural environment, which may contribute to further enhanced immunogenicity and protection. Furthermore, the other components present in the OMVs, such as for instance minor antigens including iron-regulated ones, can even further enhance the breadth of protection by the vaccines of the instant invention.

### Example 2. Coverage of MenW Strains by a Combination of PorA and FrpB Variants in OMVs According to the Invention

To determine the predicted MenW coverage of a vaccine composed of OMVs from six MenB strains having the variable regions depicted in FIG. 3, the meningococcal Molecular Epidemiology databases available at two European National Neisseria Reference Laboratories, i.e. The Netherlands (years 2002-2012) and the United Kingdom (years 2010-2014), and the international PubMLST database (years 2000-2014) were used. The two national databases were combined to yield a single database with 142 strains, while the PubMLST database with 690 international strains was used independently. Only fully fine-typed MenW strains were included.

As based on PorA VR1, PorA VR2 and FrpB VR typing, a bactericidal immune response against the strains depicted in FIG. 3, would reach ˜85% and 91% coverage of the 142 strains from the national databases and the 690 strains from the PubMLST database, respectively. In all cases PorA VR1 type P1.7-2 was excluded from the cumulative coverage calculations as this PorA VR1 type is known not to provide protection. These coverage estimates are higher compared to those reached by the combinations of six purified PorA and five purified FrpB proteins (referred to as ‘standard’ combinations in [3]) that other researchers proposed [3], [17], which would cover 71% and 88% of the isolates from the national and PubMLST databases, respectively. The coverage for the ‘enhanced’ combination of seven purified PorA and five purified FrpB antigens that these researchers proposed [3], would also be 71% and 88% for the national and pubMLST databases, respectively. This means that a higher coverage is obtained with combinations of the present invention over those described by others. In addition, compared to purified recombinant proteins, the OMV vaccines of the present invention have the additional advantage of comprising naturally folded proteins in their natural environment, which may contribute to further enhanced immunogenicity and protection. Furthermore, the other components present in the OMVs, such as for instance minor antigens including iron-regulated ones, can even further enhance the breadth of protection by the vaccines of the instant invention.

### Example 3. PorA and FrpB Overexpression in OMVs

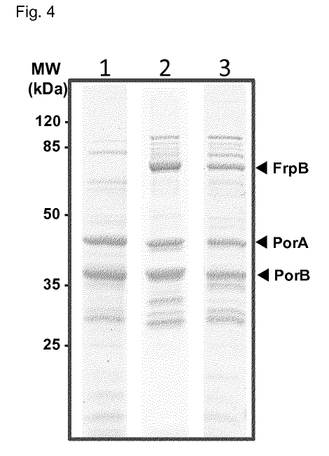

When the N. meningitidis H44/76 RLG mutant strain was grown in a shaker flask with 150 ml medium supplemented with 300 μM FeCl₃, isolated nOMVs did not show any visible FrpB on an SDS-PAGE gel (FIG. 4, lane 1).

In order to obtain nOMVs with about equal levels of the FrpB and PorA protein, the N. meningitidis H44/76 RLG mutant strain was cultured in a 5 L bioreactor with 3 L medium supplemented with 12 μM FeCl₃ and with the pH set at 7.2±0.05 (FIG. 4. lane 2). nOMV of this experiment showed that the FrpB and PorA protein content of the nOMW was 20% and 20%, respectively, of the total amount of nOMV protein.

Another method to obtain nOMVs with about equal levels of the FrpB and PorA protein was the use of the iron-chelator Desferal. When a total of 50 μM of the iron chelator Desferal was added to an early-log culture of N. meningitidis H44/76 RLG mutant strain in a shaker flask with 150 ml medium supplemented with 16 μM FeCl₃, a strong induction of FrpB expression was observed (FIG. 4, lane 3). nOMV of this experiment showed that the FrpB and PorA protein content of the nOMW was 18% and 18%, respectively, of the total amount of nOMV protein. Cell cultures in the shaker flask with and without desferal reach an optical density at 590 nm (OD590) of around 10, while the bioreactor culture with 12 μM FeCl₃ reached an OD590 of almost 8.

These results show that it possible to adjust the iron concentration in medium by lowering the iron concentration and/or the use of an iron-chelator, to obtain nOMV with about equal FrpB and PorA protein content, while it still supports growth of the culture to obtain sufficient biomass for vaccine production.

Finally, it was observed that lowering the iron concentration also induced expression of several minor outer membrane proteins with a size of 70 to 100 kDa. MS analysis confirmed that these proteins were the iron-regulated outer membrane proteins TbpA, TbpB, LbpA and HmbR.

### Example 4. Increased FrpB Expression in OMVs Improves Bactericidal Titres

To demonstrate the effect of high FrpB expression on immunogenicity of the OMV, mice were immunized with 2 doses of nOMV (5 μg total protein/dose) from the prototype strain H44/76-RLG, containing either high FrpB or low FrpB concentrations, or a buffer control.

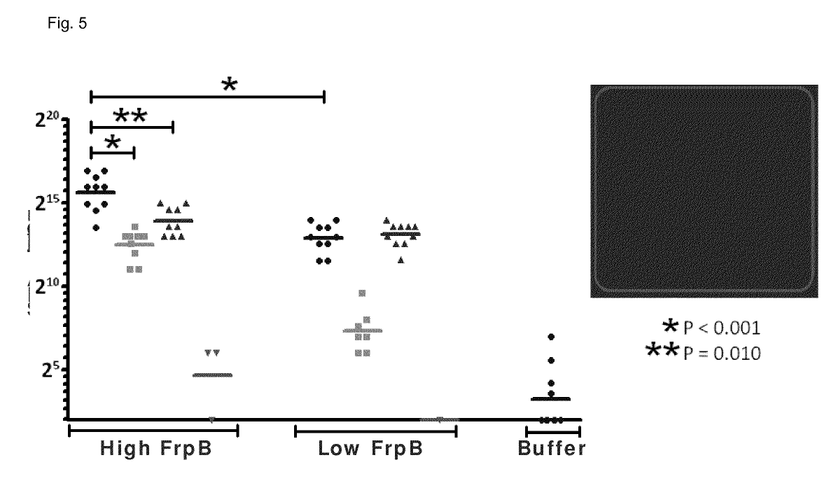

Sera from individual mice were analyzed by Serum Bactericidal Assay (SBA) against wildtype H44/76 (not RLG) and isogenic mutants: PorA-negative; FrpB-negative, and PorA+FrpB-double negative (FIG. 5).

Titres from all mice that received nOMV were very high compared to titres published in literature (for example [28] had maximum titres of 2⁹ in mice after immunization with the nOMV vaccine described therein). In our experiment, Geometric Mean Titres (GMT) against the wildtype H44/76 were 2¹⁶ after immunization with high-FrpB-nOMVs and 2¹³ after immunization with low-FrpB-nOMVs. This difference was statistically significant by Mann-Whitney 2-sample test, indicating that the increase in FrpB levels in the nOMVs leads to improved immunogenicity. GMTs of the buffer control group were low (2³), showing that the bactericidal titres measured are due to immunization with the nOMV preparations.

When low-FrpB-nOMVs were used, the GMT against the PorA-negative strain was low (2⁷) compared to the titre against the wildtype (2¹³) while the GMT against the FrpB-negative strain was also 2¹³, These data show that when nOMVs contain only low levels of FrpB, the bactericidal response generated is dominated by antibodies specific for PorA, and no bactericidal antibodies are present against FrpB.

In contrast, when high-FrpB-nOMVs were used, GMTs against the PorA-negative target strain (2¹³) and against the FrpB-negative strain (2¹⁴) were significantly lower than against the wildtype H44/76 (P<0,001 and P=0,010, respectively). Titres against the PorA/FrpB-double negative strain were low (<2⁵) in both groups. These data show that when high levels of FrpB are present in nOMVs antibodies to both PorA and FrpB contribute to bactericidal killing, and that antibodies to PorA or FrpB are required for high bactericidal killing. As such, the combination of both PorA and FrpB as major antigens in an nOMV vaccine can play an important role in increasing bactericidal titers, and therefore potential protetive efficacy, compared to a vaccine containing only one or neither of these antigens.

### Example 5. Reactogenicity and Adjuvant Activity of nOMV Preparations

To demonstrate low reactogenicity of the vaccine formulation, while maintaining the inherent adjuvant activity of nOMVs, several methods were used.

OMVs have several inherent adjuvant properties; in particular, the LipidA present in the vesicles is known to activate Toll-like Receptor (TLR) 4 on immune cells. Lipoproteins and porins in the vesicles also activate TLR2. Activation of these receptors triggers cytokine release, required for an effective immune response, via activation of NF-KB. However, activation of TLR4 by LPS in nOMVs is also associated with release of proinflammatory cytokines, associated with reactogenicity.

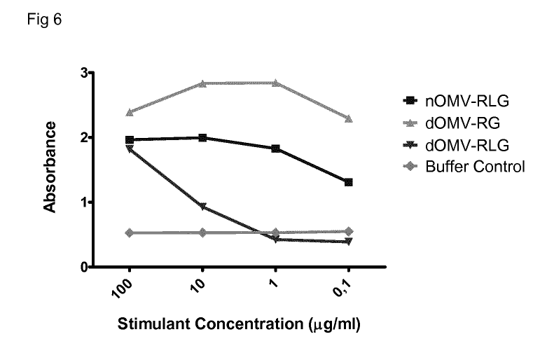

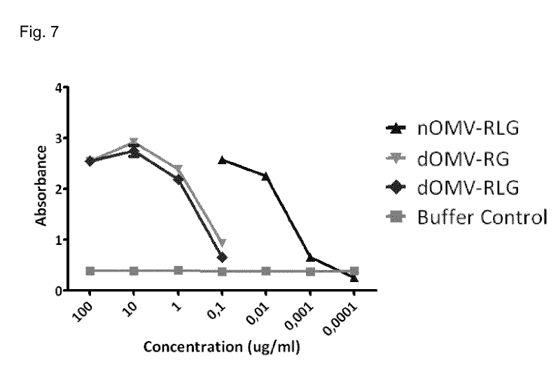

Adjuvant activity of nOMVs, via stimulation of TLR2 and TLR4, was tested using an in vitro reporter system as previously described [25]. HEK293 cells expressing either human TLR2 (+CD14) or TLR4 (+MD2, +CD14) were stimulated with nOMVs containing IpxL1 LPS (i.e. LPS from an IpxL1 mutant), or with detergent-extracted OMVs (dOMVs) containing either wildtype (dOMV-RG) or IpxL1 LPS (dOMV-RLG), each at four different concentrations. Activation of NF-κB resulting from TLR activation is measured as a colorimetric signal. Results show that nOMVs have lower TLR4-activating ability than dOMVs containing wildtype LPS. dOMVs containing IpxL1 LPS (at low levels due to removal of LPS during detergent extraction) result in the lowest TLR4-activation. The lower TLR4-stimulation with dOMV-RLG compared to dOMV-RG shows that IpxL1 LPS has a lower TLR4-stimulating activity than wildtype LPS (FIG. 6). Some TLR4-activation is still present with the nOMVs. TLR4 stimulation seen with RLG-nOMVs is higher than that seen with RLG-dOMVs as the dOMVs have undergone detergent extraction to removal much of the LPS while the nOMVs contain higher concentrations of LPS. In contrast, lower concentrations of nOMVs are required for the same level of TLR2 activation as detergent extracted vesicles (both wildtype LPS and IpxL1 LPS, FIG. 7). This is likely to be due to the presence of lipoproteins in native vesicles that are removed during detergent extraction of dOMVs, which are known to activate TLR2. From this data it can be concluded that RLG-nOMVs are less potent stimulators of TLR4 than dOMVs containing wildtype LPS, but more potent stimulators of TLR2. As TLR4 in particular is associated with the inducement of inflammatory cytokines, this reduced TLR4-stimulation could result in reduced reactogenicity while TLR2-stimulation still enables cytokine induction necessary for induction of immunity.

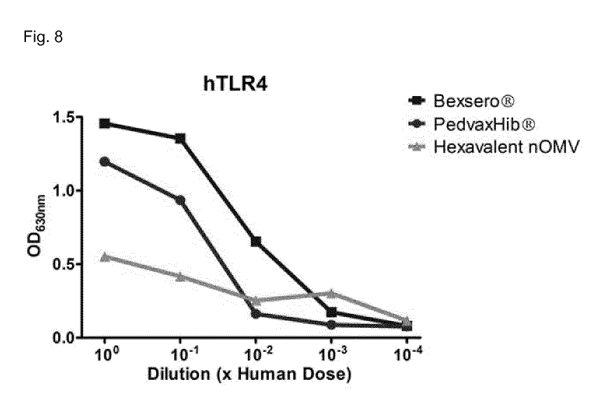

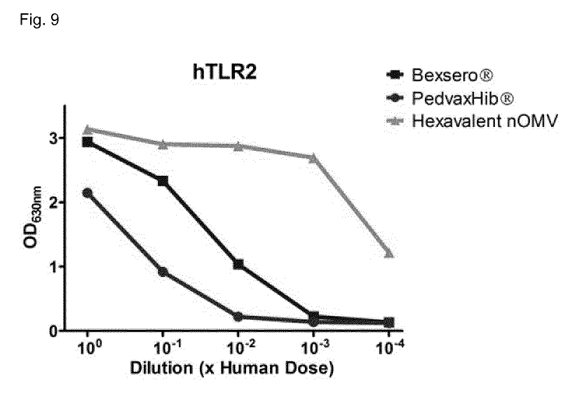

Stimulation of hTLR2 and hTLR4 by a combination of six RLG nOMVs (highest concentration tested 300 μg total protein per ml) was subsequently compared to licensed vaccines Bexsero® (Novartis Vaccines) and PedvaxHIB® (Merck Vaccines). For Bexsero® and PedvaxHIB® the highest concentration tested was one human dose. All vaccines were tested at five different concentrations. Results show that the combination nOMVs have significantly lower TLR4-stimulating activity than Bexsero® (P<0.03 by General Linear Model, GLM) (FIG. 8), which contains a dOMV with wildtype LPS. Combination nOMVs also showed significantly higher TLR2-stimulating activity compared to both Bexsero® and PedvaxHIB® (P<0.001 by GLM) (FIG. 9). PedvaxHIB® contains detergent-extracted meningococcal outer membrane proteins, and has been reported to require activation of TLR2 for optimal immunogenicity [42].

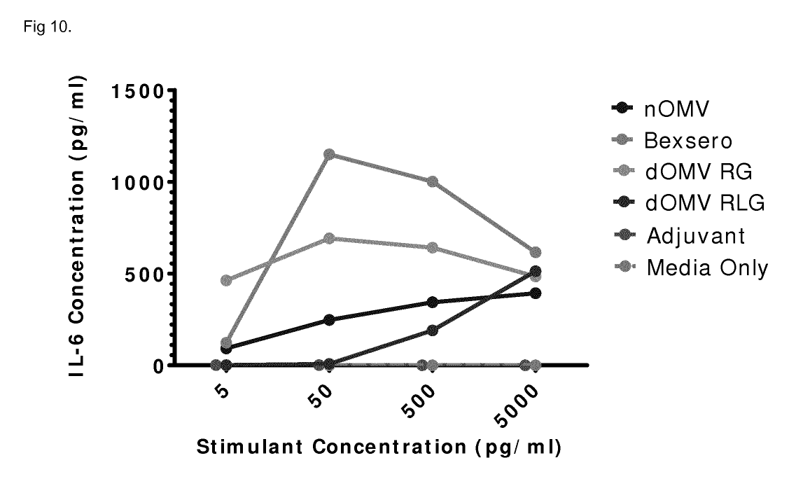

These data suggest that although TLR4 activation of the nOMVs is lower then licensed vaccines due to the IpxL1 LPS, the adjuvant activity of the vesicles is maintained by the increased activation of TLR2. The reduced activation of TLR4 is also likely to result in lower reactogenicity of the vesicles. Reactogenicity of OMVs, in particular the induction of fever following vaccination, is associated with the release of a number of inflammatory cytokines by mononuclear cells in the bloodstream. As an indicator of reactogenicity, frozen human Peripheral Blood Mononuclear Cells (PBMCs) were stimulated with nOMVs containing IpxL1 LPS, with detergent extracted OMVs containing either wildtype or IpxL1 LPS, or with Bexsero®. The induction of IL-6 was measured after 16 hours. Results (FIG. 10) show that RLG nOMVs (nOMVs from RLG strains) stimulate lower concentrations of the inflammatory cytokine IL-6 than dOMVs or Bexsero®, indicating that nOMVs (from RLG strains) are likely to have a lower reactogenicity in vivo that previously- or currently-used dOMV MenB vaccines.

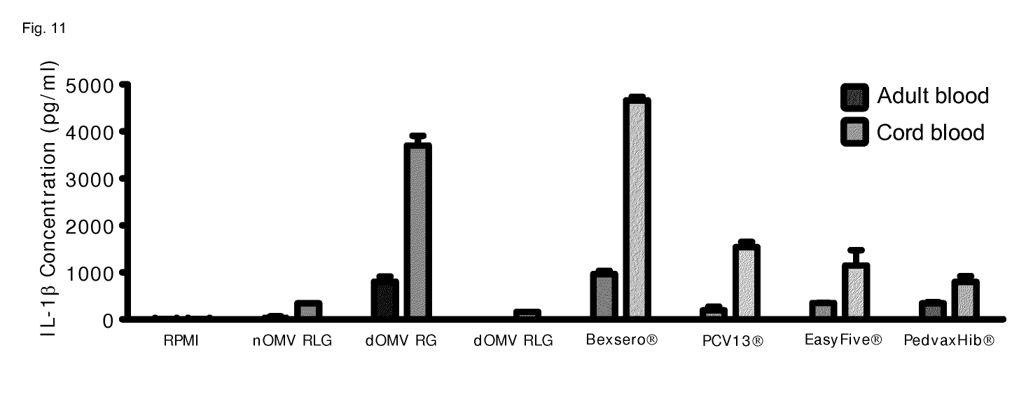

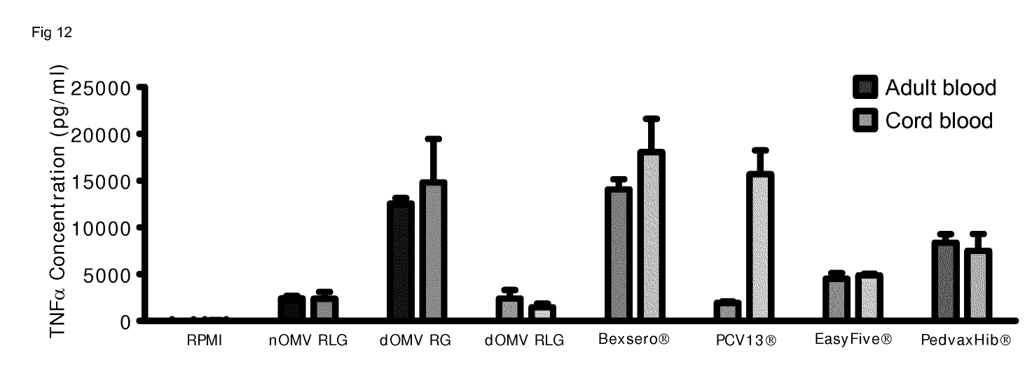

Reactogenicity of monovalent nOMVs was also compared to licensed pediatric vaccine formulations in a whole blood assay using both adult blood and cord blood. Levels of the inflammatory cytokines IL-1β(FIG. 11) and TNFα (FIG. 12) were measured by ELISA. Results show that in both adult and cord blood the RLG-nOMV formulation induces lower levels of these two cytokines than all the licensed vaccines tested. In comparison, levels of IL-1βand TNFα induced by Bexsero®, and by RG-dOMVs (i.e. dOMVs from RG strains, thus having wt LpxL1) from the same strain were high.

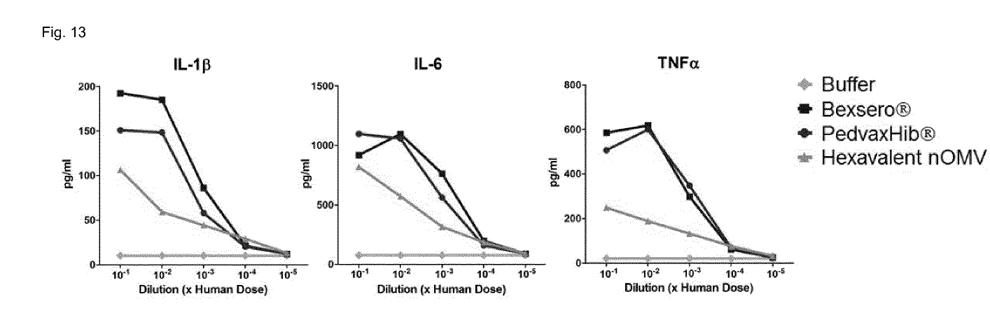

A combination of six RLG nOMVs was subsequently compared to licensed vaccines Bexsero® (Novartis Vaccines) and PedvaxHIB® (Merck Vaccines) for cytokine induction in human PMBCs as described above. For Bexsero® and PedvaxHIB® the highest concentration tested was one human dose, while for nOMVs the highest concentration tested was 300 μg total protein per ml. Results (FIG. 13) show that nOMVs induce significantly lower levels of proinflammatory cytokines IL-1βand IL-6 compared to Bexsero® (P <0.02 by GLM), and significantly lower levels of TNFa compared to both Bexsero® and PedvaxHIB® (P<0.02 by GLM).

These results collectively indicate that nOMVs are likely to have a lower reactogenicity in vivo than previously- or currently-used dOMV MenB vaccines.

### Example 6. For Particular Genotypes, RLG nOMVs can Give Higher Immunogenicity than the Corresponding Wildtype dOMVs

To demonstrate the potential coverage of a combination of OMVs, mice were immunized with equal concentrations of six RLG nOMVs or six wildtype dOMVs containing the same PorA and FrpB variants. Both OMV preparations were given at 30 μg total protein/dose. As dOMVs and nOMVs were tested in different studies, Bexsero® was used as a comparator in both studies (1/5^{th} human dose).

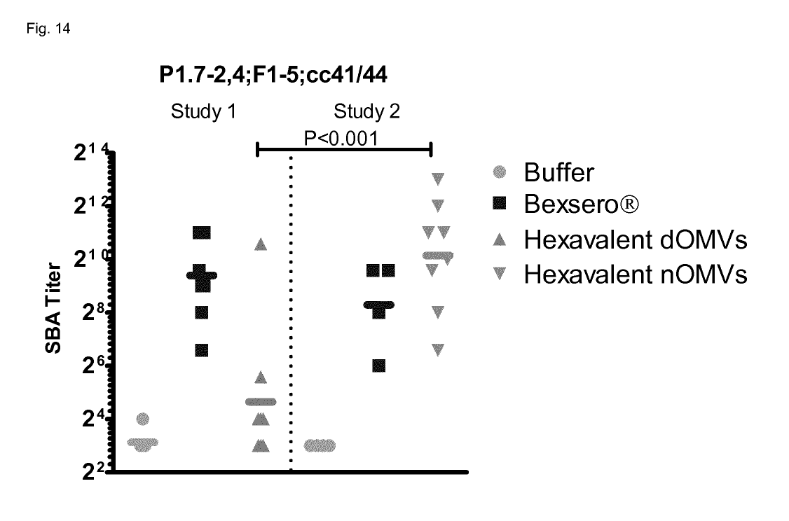

Results show that for a P1.7-2,4;F1-5 target strain, the use of a combination of nOMVs gave significantly higher SBA titers than the use of a combination of dOMVs (FIG. 14, P<0.0001 by GLM). SBA titers obtained with Bexsero® were not significantly different between the two studies (P=0.392 by GLM). The PorA variant present in this genotype (P1.7-2,4) is known to be relatively poorly immunogenic compared to other PorA variants [43]; therefore, these results suggest that for some genotypes nOMVs can be more immunogenic than isogenic dOMVs.

### Example 7. A Combination of RLG nOMVs Expressing PorA and FrpB can Provide Broader Coverage than Bexsero®

To demonstrate the potential coverage of a combination of five RLG nOMVs, mice were immunized with equal concentrations of five RLG nOMVs (either 2.5 μg each nOMV or 5 μg of each nOMV). Bexsero® was used as a comparator (1/10^{th} human dose). Sera from individual mice were tested by SBA against a panel of five MenB target strains representing five genotypes that are major causes of MenB disease worldwide.

Against these five genotypes, sera from mice given a combination of RLG nOMVs showed positive bactericidal titers against 4/5 strains. Sera from mice given Bexsero® showed positive bactericidal titers against only 3/5 strains. The target strains covered by Bexsero® in this experiment have known homology with the PorA and fHbp antigens present in the vaccine.

To demonstrate the potential coverage of a combination of six RLG nOMVs, mice were immunized with equal concentrations of six RLG nOMVs (30 μg total protein per dose). Bexsero® was used as a comparator (1/5^{th} human dose). Sera from individual mice were tested by SBA against a panel of six meningococcal target strains representing five genotypes that are major causes of meningococcal disease worldwide, including five MenB isolates and one MenW isolate.

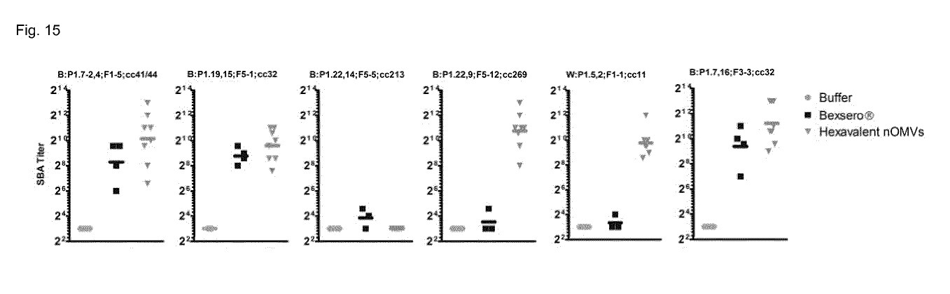

Against these six genotypes, sera from mice given a combination of RLG nOMVs showed positive bactericidal titers against 5/6 strains (FIG. 15). Sera from mice given Bexsero® showed positive bactericidal titers against only 3/6 strains, including one strain known to share homology with the PorA variant present in Bexsero® (P1.7-2,4), and two strains expressing the Factor H Binding Protein (fHbp) variant present in Bexsero® (v1.1).

For one target strain (B:P1.22,14;F5-5), only low bactericidal titers were achieved with all formulations tested. Although following immunization with the combination of RLG nOMVs, positive antigen-specific IgG was present in sera (as measured by ELISA), this did not result in positive bactericidal activity against this strain. Previous studies with target strains from this genotype also showed no or low coverage in SBA [44, 45], indicating that this genotype is likely to be fairly resistant to complement-mediated killing. Higher levels of anti-PorA and anti-FrpB IgG, which could be induced by increasing nOMV dose or formulation, may lead to improved coverage against this genotype.

Overall, immunization of mice with a hexavalent RLG-nOMV formulation resulted in broader coverage against the target strains tested than immunization with Bexsero® (coverage of 5/6 strains versus 3/6 strains).

### REFERENCES

A number of publications are cited above in order to more fully describe and disclose the invention and the state of the art to which the invention pertains. Full citations for these references are provided below. All documents mentioned in this specification are incorporated herein by reference in their entirety.
1. Cartwright et al., Vaccine 17 (1999), pp 2612-2619
2. U.S. Pat. No. 6,482,807
3. WO 2005/117956
4. http://pubmlst.org/neisseria/FetA/vr.shtml
5. http://pubmlst.org/neisseria/PorA/vr2.shtml
6. http://pubmlst.org/neisseria/PorA/vr1.shtml
7. van der Ley at al. Infection and Immunity, (October, 2001), 69: 5981-5990 (PMID:
11553534)
8. Bos and Tommassen, Infection and Immunity, (September, 2005), pp 6194-6197 Vol. 73,
No. 9 (PMID: 16113348)
9. Jennings et al., Mol Microbiol 1993, 10: 361-369 (PMID: 7934827)
10. van der Voort et al. Infection and Immunity, (July 1996), 64: 2745-2751 (PMID:
8698504).
11. van de Waterbeemd, Vaccine 2010, 28: 4810-4816 (PMID: 20483197)
12. Norheim et al (2004) Vaccine 22:2171-2180
13. WO 2013/006055
14. Pettersson et al Vaccine (2006) 24: 3545-3557
15. U.S. Pat. No. 6,558,677
16. Rappuoli et al. Vaccine. (2001) 21; 19(17-19):2688-91.
17. Unwin et al. Infection and Immunity, (2004). P5955-5962 Vol 72 No. 10.
18. Kortekaas et al. 2007 Vaccine. (January, 2007) 2; 25(1):72-84 (PMID: 16914236)
19. Delany et al 2006 J Bacteriol. (April; 188), (7):2483-92
20. van der Ley et al. Microbiology. (1996), 142, pp 3269-3274
21. Martin et al Clin Vaccine Immunol, (2006), 13(4): 486-91
22. Pettersson et al, (1990) Infect. Immun. 58(9):3036-41 (PMID 1696939)
23. Weynants et al., Infection and Immunity, (2007) p. 5434-5442, Vol. 75
24. Maslanka et al Clin. Diagn. Lab. Immunol., (1997) 4(2):156-167
25. Satta et al., (2011) Blood 117:5523-5531
26. Kikkert et al (2008) Journal of Immunological Methods 336:45-55
27. Levy et al., (2006) J. Immunol. 177:1956-1966
28. Zollinger et al (2010), Vaccine 28:5057-5067
29. Ferrari et al.(2006). Proteomics 6: 1856-1866.
30. Koeberling et al, Clin Vaccine Immunol. (May, 2011); 18(5):736-42 (PMID: 21367981)
31. Van de Waterbeemd et al, Plos One May 31, 2013, DOI: 10.1371/journal.pone.0065157 (PMID: 23741478)
32. EP2482847 B1. Purification of bacterial vesicles
33. Massari P, et al., (2006) J. Immunol. 176(4):2373-2380.
34. Al-Bader T, Jolley K A, Humphries H E et al., (2004) Cell Microbiol. 6(7):651-662.
35. Saleem et al, PLoS One.,(2013) 8(2):e56746 (PMID 23457610)
36. van der Ley et al, Vaccine (1995), 13: 401-407
37. Remington's Pharmaceutical Sciences, 18th edition, A. R. Gennaro, Ed., Mack Publishing Company [1990]; Pharmaceutical Formulation Development of Peptides and Proteins, S. Frokjaer and L. Hovgaard, Eds., Taylor & Francis [2000]; and Handbook of Pharmaceutical Excipients, 3rd edition, A. Kibbe, Ed., Pharmaceutical Press [2000]
38. van de Waterbeemd et al, (2013) J. Proteome Res 12: 1898-1908 (PMID: 23410224)
39. McNeil L K et al. Microbiol. Mol. Biol. Rev. (2013), 77(2): 234- 252.
40. Jiang H Q, et al (2010) Vaccine 28:6086 -6093.
41. Van de Waterbeemd et al, Plos One Jan. 23, 2013, DOI: 10.1371/journal.pone.0054314
42. Latz A, et al, The Journal of Immunology (2004) 172: 2431-2438.
43. Luijk T A, et al., Clin. Vacc. Immunol. (2008) 15(10): 1598-1605.
44. Findlow, J. et al., (2010) CID 51:1127-1137
45. Pinto, V. B. et al., (2011) Vaccine 29:7752-7758

Informal sequence listing

| | |
|---|---|
| SEQ ID NO: 1 (PorA VR1 P1.7-2) | AQAANGGASGQVKVTKA |
| SEQ ID NO: 2 (PorA VR1 P1.22) | QPSKAQGQTNNQVKVTKA |
| SEQ ID NO: 3 (PorA VR1 P1.19) | PPSKSQPQVKVTKA |
| SEQ ID NO: 4 (PorA VR1 P1.7) | AQAANGGASGQVKVTKVTKA |
| SEQ ID NO: 5 (PorA VR1 P1.5) | PLQNIQPQVTKR |
| SEQ ID NO: 6 (PorA VR2 P1.4) | HVVVNNKVATHVP |
| SEQ ID NO: 7 (PorA VR2 P1.14) | YVDEKKMVHA |
| SEQ ID NO: 8 (PorA VR2 P1.15) | HYTRQNNADVFVP |
| SEQ ID NO: 9 (PorA VR2 P1.16) | YYTKDTNNNLTLVP |
| SEQ ID NO: 10 (PorA VR2 P1.9) | YVDEQSKYHA |
| SEQ ID NO: 11 (PorA VR2 P1.2) | HFVQQTPKSQPTLVP |
| SEQ ID NO: 12 (FrpB F1-5) | SQFKIEDKEKATDEEKNKNRENEKIAKAYRLT |
| SEQ ID NO: 13 (FrpB F5-5) | GKFKISDKKPDPNDPTKEIDKDAAEKAKDKKDMDLVHSYKLS |
| SEQ ID NO: 14 (FrpB F5-1) | GEFEISGKKKDPKDPKKEIDKTDEEKAKDKKDMDLVHSYKLS |
| SEQ ID NO: 15 (FrpB F3-3) | SKFSIPTTEEKNGQKVDKPMEQQMKDRADEDTVHAYKLS |
| SEQ ID NO: 16 (FrpB F5-12) | GEFKISDKKPDPTDPKKEIAKTDEEKAKDKIDMDLVHSYKLS |
| SEQ ID NO: 17 (FrpB F4-1) | SKFEISDKKKGADGKEVDVDDAQKEKNRANEKIVHAYKLS |

1. A composition comprising outer membrane vesicles (OMVs) from at least six different N. meningitidis B (MenB) strains, wherein the MenB strains together comprise
a PorA variable region 2 (VR2) having the sequence set out in SEQ ID NO: 6, a PorA VR2 having the sequence set out in SEQ ID NO: 10, a PorA VR2 having the sequence set out in SEQ ID NO: 7, a PorA VR2 having the sequence set out in SEQ ID NO: 8, a PorA VR2 having the sequence set out in SEQ ID NO: 9, and a PorA VR2 having the sequence set out in SEQ ID NO: 11; and
an FrpB VR having the sequence set out in SEQ ID NO: 12, an FrpB VR having the sequence set out in SEQ ID NO: 14, an FrpB VR having the sequence set out in SEQ ID NO: 13, an FrpB VR having the sequence set out in SEQ ID NO: 16, an FrpB VR having the sequence set out in SEQ ID NO: 15, and an FrpB VR having the sequence set out in SEQ ID NO: 17.

2. The composition according to claim 1, wherein the MenB strains further comprise one or more of:
a PorA VR1 having the sequence set out in SEQ ID NO: 2, a PorA VR1 having the sequence set out in SEQ ID NO: 3, a PorA VR1 having the sequence set out in SEQ ID NO: 4, or a PorA VR1 having the sequence set out in SEQ ID NO: 5.

3. The composition according to claim 1, wherein the MenB strains further comprise a PorA VR1 having the sequence set out in SEQ ID NO: 1.

4. The composition according to any preceding claim 1, wherein each MenB strain expresses exactly one PorA VR1, one PorA VR2 and one FrpB VR.

5. The composition according to claim 1, wherein the at least six MenB strains comprise:
a strain having the PorA VR2 sequence set out in SEQ ID NO: 6 and FrpB VR sequence set out in SEQ ID NO: 12,
a strain having the PorA VR2 sequence set out in SEQ ID NO: 7 and FrpB VR sequence set out in SEQ ID NO: 13,
a strain having the PorA VR2 sequence set out in SEQ ID NO: 8 and FrpB VR sequence set out in SEQ ID NO: 14,
a strain having the PorA VR2 sequence set out in SEQ ID NO: 9 and FrpB VR sequence set out in SEQ ID NO: 15,
a strain having the PorA VR2 sequence set out in SEQ ID NO: 10 and FrpB VR sequence set out in SEQ ID NO: 16, and
a strain having the PorA VR2 sequence set out in SEQ ID NO: 11 and FrpB VR sequence set out in SEQ ID NO: 17.

6. The composition according to any preceding claim 1 wherein the at least six MenB strains comprise:
a strain having the PorA VR1 sequence set out in SEQ ID NO: 1; PorA VR2 sequence set out in SEQ ID NO: 6 and FrpB VR sequence set out in SEQ ID NO: 12,
a strain having the PorA VR1 sequence set out in SEQ ID NO: 2; PorA VR2 sequence set out in SEQ ID NO: 7 and FrpB VR sequence set out in SEQ ID NO: 13,
a strain having the PorA VR1 sequence set out in SEQ ID NO: 3; PorA VR2 sequence set out in SEQ ID NO: 8 and FrpB VR sequence set out in SEQ ID NO: 14,
a strain having the PorA VR1 sequence set out in SEQ ID NO: 4; PorA VR2 sequence set out in SEQ ID NO: 9 and FrpB VR sequence set out in SEQ ID NO: 15,
a strain having the PorA VR1 sequence set out in SEQ ID NO: 2; PorA VR2 sequence set out in SEQ ID NO: 10 and FrpB VR sequence set out in SEQ ID NO: 16,
a strain having the PorA VR1 sequence set out in SEQ ID NO: 5; PorA VR2 sequence set out in SEQ ID NO: 11 and FrpB VR sequence set out in SEQ ID NO: 17.

7. The composition of claim 1, wherein the MenB strains comprise IpxL1 and/or IpxL2 mutations.

8. The composition of claim 1, wherein the MenB strains comprise rmpM or gna33 mutations.

9. The composition of claim 1, wherein the MenB strains comprise IpxL1 and rmpM mutations.

10. The composition of claim 1, wherein the MenB strains comprise siaD and galE mutations.

11. The composition of claim 1, wherein the MenB strains overexpress one factor H-binding protein (fHpb) member of family A and one fHpb member of family B relative to wild type strain.

12. The composition of claim 11, wherein each of the MenB strains overexpresses a different fHpb family A or family B member.

13. The composition according to claim 1, wherein the composition comprises OMVs from 6-10 different MenB strains.

14. The composition according to claim 1, wherein the OMVs are native OMVs (nOMVs).

15. The composition according to claim 1, wherein the ratio of PorA to FrpB protein in the composition is between 3:1 to 1:3, optionally between 2:1 and 1:2, optionally between 1.5:1 and 1:1.5.

16. A vaccine comprising the composition according to claim 1.

17. The vaccine according to claim 16 further comprising an adjuvant.

18. (canceled)

19. A method for immunizing a subject against Neisseria meningitidis infection, the method comprising administering to said subject a vaccine according to claim 16.

20. A method for producing a N. meningitidis B (MenB) outer membrane vesicle composition according to claim 1, comprising growing the at least six MenB strains and isolating the outer membrane vesicles produced by said strains.

21. The method according to claim 20, wherein the MenB strains are grown in a chemically defined medium which has a limiting iron content to induce FrpB expression.

22. The method according to claim 21, wherein the iron content is 0-22 μM, preferably 5-20 μM or 0-2 μM for iron supplement FeCl₃ and Ferricammoniumcitrate, respectively.

23. The method according to claim 20, wherein the medium comprises an iron-chelating agent during growth of the MenB strains.

24. A composition produced by the method of claim 20.

25. A vaccine comprising the composition according to claim 24.
